(19) United States
(12) Patent Application Publication
(10) Pub. No.: US 2008/0311575 A1
(43) Pub. Date: Dec. 18, 2008

(54) Modulation of Muci-Dependent Anti-Estrogen Resistance

(76) Inventor: Donald W. Kufe, Wellesley, MA (US)

Correspondence Address: FULBRIGHT & JAWORSKI L.L.P., 600 CONGRESS AVE., SUITE 2400, AUSTIN, TX, 78701, US

(21) Appl. No.: 11/915,291

(22) PCT Filed: May 26, 2006
(86) PCT No.: PCT/US2006/020383

§ 371 Date: Jul. 30, 2008

Related U.S. Application Data
(60) Provisional application No. 60/685,052, filed on May 26, 2005.

(51) Int. Cl. C12Q 1/68

(52) U.S. Cl. 435/6

(57) ABSTRACT

The present invention provides methods for identification and use of compounds that modulate the association of MUC1 with estrogen receptors and thereby antagonize MUC1-related resistance to anti-estrogen treatment.

This application claims priority to U.S. Provisional Patent Application Ser. No. 60/685,052, filed May 26, 2005, the entire contents of which is hereby specifically incorporated by reference in its entirety.

### BACKGROUND OF THE INVENTION

The United States government may own rights in the present invention pursuant to Grant CA097098 awarded by the National Cancer Institute and Grant BC022158 awarded by the US Army.

A. FIELD OF THE INVENTION

The present invention relates generally to the field of cancer therapy and more specifically to the identification and use of compounds that modulate the association of MUC1 with estrogen receptors and thereby antagonize MUC1-related resistance to anti-estrogen treatment.

B. RELATED ART

The MUC1 transmembrane glycoprotein is normally expressed on the apical borders of secretory mammary epithelia (Kufe, et al., 1984). With transformation and loss of polarity, MUC1 is aberrantly overexpressed in the cytosol and on the entire surface of breast cancer cells (Kufe, et al., 1984; Perey, et al., 1992). The MUC1 locus has been mapped to human chromosome 1q21 in a region that is frequently affected by genetic alterations in breast and other carcinomas (Merlo, et al., 1989; Swallow, et al., 1987). MUC1 is expressed as a stable heterodimer following translation of a single polypeptide and cleavage into two subunits in the endoplasmic reticulum (Ligtenberg, et al., 1992). The MUC1 N-terminal subunit (MUC1 N-ter, MUC1-N) contains variable numbers of 20 amino acid tandem repeats that are extensively modified by O-linked glycans (Gendler, et al., 1988; Siddiqui, et al., 1988). The MUC1 C-terminal subunit (MUC1 C-ter, MUC1-C) consists of a 58 amino acid extracellular domain, a 28 amino acid transmembrane domain and a 72 amino acid cytoplasmic tail (Merlo, et al., 1989). On the cell surface, MUC1-N extends well beyond the glycocalyx and is tetliered by MUC1-C to the cell membrane. MUC1-C also accumulates in the cytosol of transformed cells and is targeted to the nucleus (Li 2003a; Li, et al., 2003b; Li, et al., 2003c; Wen, et al., 2003) and mitochondria (Ren, et al., 2004). The MUC1 cytoplasmic domain (CD) associates with members of the catenin family (Li and Kufe 2001; Yamamoto, et al., 1997) and with the p53 tumor suppressor (Wei, et al., 2005). MUC1-CD is also subject to phosphorylation by the epidermal growth factor receptor (EGFR) (Li, et al., 2001b), c-Src (Li 2001 a) and glycogen synthase kinase 3b (GSK3b) (Li, et al., 1998). The finding that MUC1interacts with ErbB2 has further supported a role for MUC1 in both the ErbB receptor tyrosine kinase and Wnt signaling pathways (Li, et al., 2003c; Schroeder, et al., 2001). Of potential importance to the aberrant regulation of MUC1 in breast carcinomas, other studies have shown that MUC1 overexpression is sufficient to confer anchorage-independent growth and tumorigenicity (Huang, et al., 2003; Li 2003b; Schroeder, et al., 2004).

Most human breast cancers are estrogen dependent and their treatment with estrogen antagonists, particularly tamoxifen (TAM), has had a dramatic effect on mortality (Ali and Coombes 2002). Estrogen action is mediated by two members of the nuclear receptor family, estrogen receptor α (ER α) and ERβ. Both ERs contain a central DNA-binding domain (DBD), which binds to estrogen response elements (EREs), and a C-terminal ligand binding domain (LBD). ERα and ERβ have substantial homology in their DBDs and thus may regulate common sets of genes. However, in contrast to ERβ knockout mice, ERα knockout mice are infertile, supporting different roles for these receptors (Krege, et al., 1998; Lubahn, et al., 1993). ERα occupies the promoters of estrogen-responsive genes in the absence of estrogen stimulation (Metivier, et al., 2003; Metivier, et al., 2002; Reid, et al., 2003; Shang, et al., 2000). Moreover, upon estrogen binding, ERα undergoes conformational changes and dimerization that increase binding to EREs. Activation of ER a -mediated transcription is regulated by activation function-1 (AF-1) in the N-terminal region and AF-2 in the LBD. AF-1 is activated by ErbB receptor signaling and the MAP kinase pathway, and AF-2 is required for estrogen-dependent transactivation. In the response to estrogen, ER α transcription complexes on target promoters recruit coactivators from i) the pl60 family (SRC-1/NCoA-1, GRIP1/NCoA-2 and AIB1/RAC3/ACTR) (Chen, et al., 1997; Halachmi, et al., 1994; Onate, et al., 1995), ii) non-pl60 proteins (RIP140, mSUG1 and TIF1) (Cavailles, et al., 1995; Le Douarin, et al., 1995; vom Baur, et al., 1996), and iii) histone acetylases (p300 and CBP) and the p300/CBP-associated factor pCAF (Chakravarti, et al., 1996). The structural changes induced by binding of estrogen to the ERα LBD promotes the recruitment of p160 coactivators (Shiau, et al., 1998). ERα also interacts with basal transcription factors to increase the initiation of transcription (Ing, et al., 1992; Jacq, et al., 1994). Notably, recruitment of pl60 coactivators is sufficient for ERa-mediated gene activation and for estrogen-induced growth stimulation (Shang, et al., 2000). In contrast to estrogen-induced recruitment of transcriptional coactivators, TAM recruits corepressors to the ERα transcription complex and thereby blocks growth and survival (Brzozowski, et al, 1997; Halachmi, et al., 1994; Shang, et al., 2000). Nonetheless, many breast cancers become resistant to TAM, often despite continued ERα expression and by mechanisms not clearly understood.

### SUMMARY OF THE INVENTION

The present invention provides methods for identifying compounds that modulate the association of MUC1 with estrogen receptors. One aspect of the present invention are methods for screening compounds for the ability of modulating the association of a MUC1 cytoplasmic domain polypeptide with an estrogen receptor comprising: providing a first polypeptide comprising SEQ ID NO: 7 or a fragment thereof, capable of binding to an DNA binding domain; providing a second polypeptide comprising SEQ ID NO: 9, SEQ ID NO: 11, or a fragment thereof, capable of binding to MUC1 CD; providing a candidate compound; quantifying the association between said first and said second polypeptide; and comparing said quantification of the association between said first and said second polypeptide with an appropriate control, such as the binding in the absence of a test compound.

In some embodiments, the first polypeptide or the second polypeptide is immobilized by linkage to a stationary phase. In some embodiments, the first polypeptide and/or the second polypeptide further comprises a fluorescent label, a radiolabel, or a chromophore. In some embodiments, the first polypeptide and/or second polypeptide is a fusion protein. In other embodiments, the method further comprises providing 17β-estradiol in an amount sufficient to enhance the association of said first and said second polypeptide.

Another aspect of the invention is a method of screening for a compound that specifically binds to a polypeptide comprising SEQ ID NO: 7 or a fragment thereof, capable of binding to an estrogen receptor DNA binding domain, the method comprising: a) combining said polypeptide with at least one test compound under suitable conditions, and b) detecting binding of the test compound to polypeptide, thereby identifying a compound that specifically binds to said polypeptide.

Another aspect of the invention is a method for screening of compounds effective for preventing or inhibiting anti-estrogen resistance comprising identifying a compound that decreases the association of the MCU1 cytoplasmic domain with an estrogen receptor.

A further aspect of the invention is the use of all or part of the MUC1 cytoplasmic domain in a method for detecting compounds for the prevention and/or treatment of anti-estrogen resistance in breast cancer patients. In some embodiments, the method comprise: providing a first polypeptide comprising all or part of the MUC1 cytoplasmic domain capable of binding to an ER DNA binding domain; providing a second polypeptide comprising the DNA binding domain of an estrogen receptor; providing a test compound; quantifying the association between said first and said second polypeptide; and comparing said quantification of the association between said first and said second polypeptide with an appropriate control.

The terms “preventing, “inhibiting,” or “reducing,” or any variation of these terms, when used in the claims and/or the specification includes any measurable decrease or complete prevention, reduction, or inhibition to achieve a desired result. “Inhibiting,” and “preventing” does not require complete inhibition nor prevention of the association of MUC1 CD with a ER or of MUC1-dependent anti-estrogen resistance.

The use of the word “a” or “an” when used in conjunction with the term “comprising” in the claims and/or the specification may mean “one,” but it is also consistent with the meaning of “one or more, “at least one,” and “one or more than one.” It is specifically contemplated that any embodiments described in the Examples section are included as an embodiment of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. MUC1 associates with ERα. FIGS. 1A and B. Human MCF-7 (A) and ZR-75-1 (B) breast cancer cells were grown in phenol red-free medium supplemented with 10% charcoal-dextran-stripped FBS for 3 d. The cells were then left untreated or stimulated with 100 nM E2 for 3 h. Lysates were subjected to immunoprecipitation (IP) with anti-ERα or a control IgG. The immunoprecipitates were analyzed by immunoblotting (IB) with anti-MUC1-C and anti-ERa. FIGS. IC and D. COS-1 cells expressing Myc-MUC1-CD and ERα were stimulated with 100 nM E2 for 3 h. Anti-ERα (C) or anti-Myc (D) IPs were immunoblotted with anti-MUC1-C or anti-ERα. Lysates not subjected to IP were immunoblotted with anti-MUC1-C or anti-ERα (lower panels).

FIGS. 2A-D. MUC1-CD binds directly to the ERα DNA binding domain FIG. 2. A. Schema depicting the structures of MUC1-CD and ERα. Also shown for MUC1-CD are the b-catenin binding motif (boxed) and the c-Src, GSK3b and PKCd phosphorylation sites. H: hinge region. FIGS. 2B-D. GST and GST-MUC1-CD(1-72) were bound to glutathione agarose and incubated with ³⁵S-labeled ERα or the indicated ERα deletion mutants FIG. 2(B). GST, GST-MUC1-CD(1-72) or the indicated GST-MUC1-CD deletion mutants bound to glutathione agarose were incubated ³⁵S-labeled ERα FIG. 2(C). GST-MUC1-CD was incubated with the indicated ³⁵S-labeled ERα proteins in the absence of ligand (Control) and in the presence of 100 nM E2 or 100 nM TAM FIG. 2(D). After washing, bound proteins were eluted and separated by SDS-PAGE. The gels were fixed, dried and subjected to phosphoimager analysis.

FIGS. 3A-F. MUC1 occupancy of estrogen-responsive gene promoters. FIGS. 3A and B. Cells were grown in phenol red-free medium supplemented with 10% charcoal-dextran-stripped FBS for 3 d. Following treatment with 100 nM E2 for 1 h, cells were cross-linked with 1% formaldehyde and monitored by ChIP assays. Soluble chromatin from control and E2-treated MCF-7 or ZR-75-1 cells was immunoprecipitated with anti-MUC1-C or a control IgG. The final DNA extractions were amplified by PCR using pairs of primers that cover the indicated EREs or control regions (CRs) of the pS2 (A) and cathepsin D (B) gene promoters. FIGS. 3C and D. In Re-ChIP experiments, soluble chromatin from the indicated cells was immunoprecipitated with anti-MUC1-C. The immune complexes were eluted by incubation with 10 mM DTT for 30 min at 37° C. After centrifugation, the supernatant was diluted 30 times with Re-ChIP buffer, followed by reprecipitation with anti-ERα and then detection of the indicated EREs or CRs in the pS2 (C) and cathepsin D (D) gene promoters. FIG. 3E. MCF-7 and ZR-75-1 cells were treated with 100 nM E2 for the indicated times. Soluble chromatin was immunoprecipitated with anti-MUC1-C or anti-ERα and analyzed for pS2 and cathepsin D ERE sequences.

FIGS. 4A-D. MUC1 increases ERα occupancy and coactivates ERaα-mediated transactivation. FIG. 4A. MCF-7/CsiRNA and MCF-7/MUC1siRNA-A cells were treated with 100 nM E2 for 1 h. Soluble chromatin was immunoprecipitated with anti-MUC1-C and analyzed for pS2 and cathepsin D ERE sequences. FIG. 4B. MCF-7/MUC1siRNA-A (open bars) and MCF-7/CsiRNA (solid bars) cells were transfected with 500 ng ERE-tk-Luc (Chen et al., 1999), an internal control LacZ expression plasmid (pCMV-LacZ) and the indicated amounts of an ERa expression vector. At 18 h after transfection, the cells were left untreated or stimulated with 100 nM E2 for 24 h. Luciferase activity was normalized to that obtained for LacZ and is presented as relative luciferase activity (mean+SD of 3 separate experiments) compared to that obtained with FIG. 4 the E2-stimulated MCF-7/MUC1siRNA cells (open bar; normalized to 1) in lane 2. FIG. 4C. ZR-75-1/vector and ZR-75-1/MUC1siRNA cells were treated with 100 nM E2 for 1 h. Soluble chromatin was immunoprecipitated with anti-MUC1-C and analyzed for pS2 and cathepsin D ERE sequences. FIG. 4D. ZR-75-1MUC1siRNA (open bars) and ZR-75-1/vector (solid bars) cells were transfected with ERE-tk-Luc, pCMV-LacZ and ERα as indicated, stimulated with E2 and analyzed for luciferase activity as described for MCF-7 cells in B.

FIGS. 5A-D. MUC1 stabilizes ERα. FIG. 5A. Lysates from the indicated MCF-7 (left) and ZR-75-1 (right) cells were immunoblotted with anti-ERa, anti-MUC1-C and anti-β-actin. WT: wild-type cells. FIGS. 5B and C. The indicated MCF-7 (left) and ZR-75-1 (right) cells were treated with 5 mM MG132 for 24 h. Lysates were subjected to immunoblotting with the indicated antibodies FIG. 5B. Anti-ERα immunoprecipitates were analyzed by immunoblotting with anti-Ub or anti-ERα FIGS. 5C and D. MCF-7/CsiRNA (n) and MCF-7/MUC1siRNA (o) cells were pulsed with [³⁵S]-methionine, washed and incubated in the presence of 10 nM E2 for the indicated times. Lysates were immunoprecipitated with anti-ERα and the precipitates were analyzed by SDS-PAGE and autoradiography. A higher amount of MCF-7/MUC1siRNA cell lysate was used for immunoprecipitation to increase the ERα signals. Lysates not subjected to immunoprecipitation were immunoblotted with anti-β-actin. Intensity of the signals as determined by densitometric scanning is presented as the percentage of ERα remaining over time relative to control at 0 h.

FIGS. 6A-E. MUC1 coactivates ERa-mediated gene transcription. FIGS. 6A and C. Soluble chromatin from the indicated cells left untreated, treated with 100 nM E2 for 1 h or treated with 1 mM TAM for 1 h was immunoprecipitated with anti-SRC-1 or anti-GRIP1 and analyzed for pS2 (left) and cathepsin D (right) gene promoter sequences. FIGS. 2B and D. MCF-7/MUC1siRNA-A (FIG. 6B; open bars), MCF-7/CsiRNA (FIG. 6B; solid bars), ZR-75-1/MUC1siRNA (FIG. 6D; open bars) and ZR-75-1/vector (FIG. 6D; solid bars) cells were transfected with 500 ng ERE-tk-Luc and 10 ng pCMV-LacZ. At 18 h after transfection, the cells were left untreated or stimulated with 100 nM E2 in the absence or presence of 1 mM TAM for 24 h. Relative luciferase activity is presented as the mean+SD of 3 separate experiments compared to that obtained with the E2-stimulated MUC1-negative cells (open bar; normalized to 1). FIG. 6E. The indicated cells were left untreated, treated with 100 nM E2 or 1 mM TAM for 24 h. Lysates were subjected to immunoblotting with the indicated antibodies.

FIGS. 7A-D. MUC1 attenuates anti-estrogen-induced loss of survival. FIG. 7A. MCF-7 cells were grown in DMEM medium supplemented with 10% FBS and increasing concentrations of TAM (0.05 to 1 mM) for 12 months. Lysates were immunoblotted with the indicated antibodies. FIG. 7B. Soluble chromatin from the indicated MCF-7 cells was immunoprecipitated with anti-MUC1-C or anti-ERα and analyzed for pS2 (left) and cathepsin D (right) promoter sequences. FIGS. 7C and D. The indicated cells were grown (500/well) in 6-well plates containing 2 ml/well phenol red-free medium supplemented with 10% charcoal-dextran-stripped FBS for 24 h at 370C. The cells were then cultured for 10 d in the presence of 100 nM E2, 1 mM TAM or 100 nM ICI. Colonies were stained with crystal violet and counted manually. The results are expressed as percentage colony formation (mean+SD of 3 separate experiments) compared to that obtained with untreated cells.

FIG. 8. Scheme depicting the proposed interactions between MUC1 and ERα.

### DETAILED DESCRIPTION OF THE INVENTION

A. The Interaction between MUC1 and ER

MUC 1 is aberrantly overexpressed by most human breast carcinomas (Kufe, et al., 1984). The MUC1-N subunit is tethered to MUC1-C at the cell membrane. MUC-C also accumulates in the cytosol and is targeted to the nucleus or mitochondria (Li, et al., 003a; Li, et al., 2003b; Li, et al., 2003c; Ren, et al., 2004; Wen, et al., 2003). The present invention demonstrate that MUC1-C associates with ER and that the binding of MUC1 and ERα was detectable constitutively and increased in response to E2 stimulation. The results further demonstrate that MUC1 is present with the ERα transcription complex on promoters of the estrogen-responsive pS2 and cathepsin D genes. MUC1 polypetides, such as amino acids 9-46 in the cytoplasmic domain, bind directly to the ERα DBD and increases ERa occupancy of estrogen-responsive promoters.

ERα is degraded by the ubiquitin-proteosome pathway in both the absence and presence of ligand (Lonard, et al., 2000; Nawaz, et al., 1999; Reid, et al., 2003). Moreover, recruitment of E3 ligases and subunits of the proteosome confers cyclic turnover of ERα on estrogen responsive promoters (Reid, et al., 2003). The present results demonstrate that MUC1 stabilizes ERα by attenuating its ubiquitination and degradation. Thus, ERα levels are substantially higher in MCF-7 and ZR-75-1 cells as compared to that following stable silencing of MUC1 expression. ERα occupancy of the pS2 and cathepsin D promoters was also increased by a MUC1-dependent mechanism, consistent with decreased turnover of ERα on responsive genes by the ubiquitin-proteosome pathway. The E3 ligases Mdm2 and E6AP are recruited to the pS2 promoter and therefore may contribute to the ubiquitination of unliganded and liganded ERα (Reid 2003). The inventors believe the present invention provides the first evidence for a protein that interacts directly with ERα and contributes to ERα stabilization.

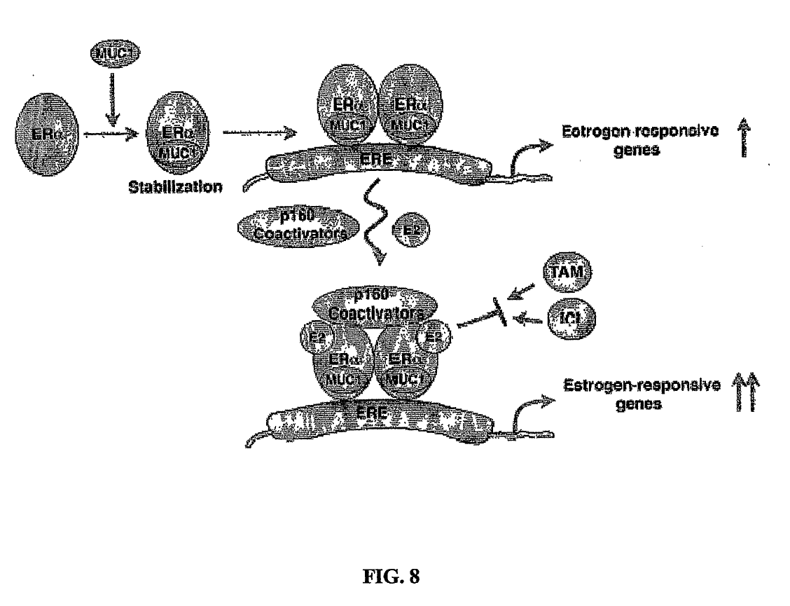

The present invention demonstrates that MUC1 expression is associated with resistance to anti-estrogens. TAM competes with E2 for binding to ERα and induces conformational changes in the ERα AF2 domain that block recruitment of p160 coactivators (Brzozowski, et al., 1997; Halachmi, et al., 1994). The results demonstrate that MUC1 attenuates TAM-induced decreases of SRC-1 and GRIP1 in the ERα transcription complex. In concert with these findings, MUC1 also attenuated TAM-induced repression of ERα -mediated transcription. TAM induces binding of the nuclear matrix protein/scaffold attachment factor HET/SAF-B to the ERα DBD and thereby inhibits ERα -mediated transactivation (Oesterreich, et al., 2000). Thus, binding of MUC1 to the ERa DBD could interfere with TAM-induced interactions between ERα and HET/SAF-B. Other mechanisms have been associated with resistance to TAM action. Upregulation of coactivators and/or downregulation of corepressors can contribute to ERα activation in cells treated with TAM (Smith, et al., 1997; Webb, et al., 1998). Certain mutations in ERα can also contribute to ERα activation in the presence of TAM (Zhang, et al., 1997). Other studies have indicated that activation of ErbB receptor signaling pathways and thereby phosphorylation of the ERα AFI domain can confer TAM resistance (Benz, et al, 1993; Kurokawa, et al., 2000; Nicholson, et al., 2001). In this regard, MUC1 functions as a coreceptor for EGFR and ErbB2 (Li, et al., 2001b; Li, et al., 2003c; Schroeder, et al., 2001); thus, the present results do not exclude the possibility that, in addition to direct binding to the ERα DBD, MUC 1 may also contribute to TAM resistance through ErbB signaling pathways. The results further indicate that MUC1 can block TAM-induced death of breast cancer cells. In both cell types, TAM had limited effects (˜10% or less) on the induction of apoptosis (data not shown). Colony assays were therefore performed as a more robust measure of the effects of MUC 1 on TAM-induced death. Consistent with the demonstration that MUC1 attenuates the effects of TAM on recruitment of p160 coactivators and ERα-mediated transcription, MUC1 expression was associated with attenuation of TAM-induced loss of clonogenic survival. MUC1 also attenuated ICI-induced turnover of ERα and ICI-induced cell death. These findings indicate that MUC1 confers resistance to anti-estrogens. While not being bound by any particular theory, FIG. 8 provides a diagrammatic representation of the proposed interactions of MUC1 with ERα.

Stabilization of ERα as conferred by MUC1 could thus contribute to growth stimulation and survival of the 90% of human breast cancers that overexpress MUC1. Most patients with ERa-positive metastatic breast cancer either fail to respond to anti-estrogens or develop resistance to anti-estrogen therapy, making such failure a major obstacle for breast cancer treatment. The present invention provides the first evidence that overexpression of MUC1 as found in most human breast cancers could contribute to resistance to anti-estrogens. These findings provide the basis for developing strategies that overcome this potential mechanism for failure of anti-estrogen therapy.

Thus, the present invention provides for methods of screening candidate compounds for the ability to modulate the interaction between MUC1 and ER. Such agents that inhibit the MUC1/ER interaction will prevent or diminish MUC1-dependent anti-estrogen resistance. Such agents may be used to treat individuals who have developed anti-estrogen resistance, used prophylactically to prevent or diminish the development of such resistance or otherwise increase the responsiveness to anti-estrogen treatment.

### B. Hormonal Therapy

The fundamental treatment choice for the majority of advanced breast cancer (ABC) patients is between cytotoxic chemotherapy and endocrine therapy. For patients with hormone-receptor-positive breast cancer, endocrine therapy is the preferred treatment. Chemotherapy causes tumor regression more quickly than endocrine therapy, but the advantage of endocrine therapy, however, is that it offers antitumor activity without the detrimental adverse events associated with cytotoxic chemotherapy that lead to a significantly reduced quality of life.

The most important indicator of response to endocrine therapy is the presence of estrogen receptors (ERs) and progesterone receptors (PgRs) in the tumor. While endocrine therapy produces responses in approximately 30% of unselected patients, in ER-positive and/or PgR-positive patients, response rates>80% have been observed (Buzdar & Hortobágyi 1998; Ravdin, et al., 1992).

Since the 1970s, tamoxifen has been the most commonly used endocrine agent for the first-line treatment of postmenopausal metastatic breast cancer. Tamoxifen is a selective ER modulator (SERM) that competitively inhibits estradiol binding to the ER (Jordan & Dowse 1976), and in so doing, disrupts a series of cellular mechanisms that regulate cellular replication. The disruption caused by tamoxifen changes the growth factor profile in responsive tissues and causes cells to be held at the G₁ phase of the cell cycle (Osborne, et al., 1983; Colletti, et al., 1989). This produces changes in tumor cell proliferation and cell death, the balance of which results in the observed antitumor responses (Cameron, et al., 2000; Cameron, et al., 2001) and improvement in overall survival (Yao & Jordan 1998).

The levels of expression of ER and PgR have been shown to correlate with overall response to tamoxifen. Postrnenopausal patients have higher levels of ER and PgR expression than their premenopausal counterparts. For those patients with high ER and PgR expression levels, the overall response rate is as high as 70% (Ravdin, et al., 1992; Buzdar, et al., 1998). Tamoxifen has been shown to be better tolerated than—and to provide benefit equivalent to—hypophysectomy and aminoglutethimide (Kiang, et al., 1980), while being superior to standard-dose progestin therapy (Muss, et al, 1988). Eventually, disease relapse and resistance to tamoxifen treatment develop in many patients. The compounds provided by the present invention are useful in the prevention or treatment of anti-estrogen treatment in patients in need of such treatment.

ICI 182,780 is a highly specific ER antagonist marketed under the trade name Falsodex (Howell, et al., 2000). ICI 182,780 is used for treating ER-dependent tumors.

### C. MUC1 Proteins and Polypeptides.

The “full-length” MUC 1 protein exhibits a variable number of tandem repeats and can thus vary in size. Consequently, the 1255 amino acid sequence of SEQ ID NO: 1 is representative of a protein with a range of sequence lengths, e.g., GenBank A35175[gi:11385307] (1344 amino acid residues) and A35887[gi:107111] (1335 amino acid residues).

A number of MUC1 splice variants have been described, including several that retain the transmembrane domain. SEQ ID NO: 2 represent a 255 amino acid sequence that is the equivalent of residues ¹M to ⁵³A and ¹⁰⁵⁴F to ¹²⁵⁵L of “full-length” MUC1 (SEQ ID NO: 1). This sequence has been termed MUC1/Y (Zirhan-Licht, et al., 1994; GenBank S48146[gi:1085342]). SEQ ID NO: 3 represents a 273 amino acid sequence that is the equivalent of ¹M to ⁵³A and ¹⁰³⁶L to ¹²⁵⁵L of SEQ ID NO: 1. This sequence has been called both MUC1/Z (Oosterkamp, et al., 1997; GenBank AAD10858[gi:4204967]) and MUC1/X (Baruch 1997). SEQ ID NO: 4 represents a 240 amino acid sequence that is the equivalent of ¹M to ⁴⁶P and 1062S to ¹²⁵⁵L of SEQ ID NO:1. This sequence has been termed MUC1/V (WO9603502). SEQ ID NO: 5 represents a 230 amino acid sequence that is the equivalent of ¹M to ⁵³A of SEQ ID NO: 1, a 17 amino acid sequence IPAPTTTIKSCRETFLKW, and ¹⁰⁹⁶P to ¹²⁵⁵L of SEQ ID NO:1. The sequence represented by SEQ ID NO:5, has been called MUC1/X (GenBank AAD10856[gi:4204963]. For the purposes of the current invention, the term “MUC1” generically applies to all variants of MUC1 that contain a transmembrane domain and the cytoplasmic domain (CD).

SEQ ID NO: 6 represents the 72 amino acids of the CD. SEQ ID NO: 7 represents a 30 amino acid fragment of the CD that binds to the ER DNA binding domain. Embodiments of the present invention include amino acid sequences comprising MUC1 CD fragments wherein such fragments may have 1, 2, 3, 4, 5, 6, 7, 8, 9, 10, 11, 12, 13, 14, 15, 16, 17, 18, 19, 20, 21, 22, 23,24, 25, 26, 27, 28,29 or 30 consecutive amino acids deleted from the amino terminal end of SEQ ID NO: 6 and wherein such fragments may have 1, 2, 3, 4, 5, 6, 7, 8, 9, 10, 11, 12, 13, 14, 15, 16, 17, 18, 19, 20, 21, 22, 23,24, 25, 26, 27, 28, 29, 30, 31, 32, 33, 34, or 35 consecutive amino acids deleted from the carboxy terminal end of SEQ ID NO: 6. Thus, SEQ ID NO: 7 represents a fragment wherein 8 consecutive amino acids have been deleted from the amino terminal end of SEQ ID NO: 6 and 26 consecutive amino acids have been deleted from the carboxy terminal of SEQ ID NO: 6.

Polypeptides or peptides utilized in the present invention may be made by any technique known to those of skill in the art, including the expression by standard molecular biological techniques, the isolation of polypeptides from natural sources, or the chemical synthesis of polypeptides. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or as would be known to those of ordinary skill in the art. Alternatively, various commercial preparations of proteins, polypeptides and peptides are known to those of skill in the art.

The ability of MUC1 CD fragments to bind to ER may be performed as generally described throughout this instant specification and more specifically as exemplified in Example 3.

### D. Estrogen Receptors

The biological actions of estrogens are mediated by binding to one of two specific estrogen receptors (ERs), ERα or ERβ, which belong to the nuclear receptor (NR) superfamily, a family of ligand-regulated transcription factors (Pettersson & Gustafsson, 2001). Ligand-binding induces conformation changes in the receptor leading to dimerization, protein-DNA interaction, recruitment of coregulator proteins, and other transcription factors, and ultimately the formation of the preinitiation complex (Rachez & Freedman, 2001) . ERs regulate gene expression by binding to their cognate response element or through protein-protein interactions with other transcription factors (Paech, 1997).

ERα and ERβ contain the evolutionarily conserved structural and functional domains typical of NR family members, including domains involved in DNA-binding, dimerization, ligand binding, and transcriptional activation (Nilsson et al, 2001). ERα and ERβ share a high degree of sequence identity within their DNA-binding domains (DBDs). The amino acid sequence of the P-box, a motif within the DBD critical for receptor-DNA recognition and specificity, is identical between the two receptors. Thus, both receptors bind estrogen responsive elements (EREs) with similar specificity and affinity, although differential subtype affinities and responses to a subset of natural EREs have been described (Klinge et al., 2001). The ligand-binding domains (LBDs) are also conserved and both receptors exhibit similar affinities for the endogenous estrogen, 17β-estradiol (E2). However, ERα and ERβ exhibit different affinities for some natural compounds and novel subtype-specific ligands have been reported (Kuiper et al., 1998; Sun et al., 1999). Characterization of mice lacking either ERα, or ERβ, or both has demonstrated that each subtype has similar but also unique roles in estrogen action in vivo. Both ERs are widely distributed throughout the body, displaying distinct but overlapping expression patterns in a variety of tissues (Pettersson & Gustafsson, 2001; Couse & Korach, 1999).

NR DBDs consists of a highly conserved core with two asymmetric zinc fingers and an ˜30 amino acid segment, termed the C-terminal extension (CTE). Within the core DBD, α-helix 1 extends between the two zinc fingers and makes base specific contacts in the major groove of the response element. The second α-helix (helix 2) does not contact DNA but is important for the overall folding of the core DBD (Schwabe 1993). The CTE is not conserved and adopts different structural motifs dependent on the class of nuclearreceptor (Khorasanizadeh & Rastinejad 2001; Senkus & Edwards 1996). Nonetheless, the CTE of different receptors does appear to share a functional role to stabilize the receptor-DNA complex by extending the protein-DNA interface beyond that of base-specific contacts made by the core DBD.

The transactivating functions of ERα and ERβ are mediated by two separate but not mutually exclusive transcription activation functions (AFs) that allow the receptors to stimulate the transcription of estrogen-regulated genes: an N-terminal ligand-independent activation function (AF-1), and a C-terminal ligand-dependent activation function (AF-2) located within the LBD (Nilsson, et al., 2001). The AFs contribute to estrogen-mediated transcription and mediate cell- and promoter-specificity. A comparison of the AF-1 domains of the two ERs has revealed that this domain is very active in ERα on a variety of estrogen responsive promoters, but under identical conditions, the activity of AF-1 in ERP is minimal (Barkhem, et al., 1998). These two receptors also exhibit distinctive responses to the synthetic antiestrogens tamoxifen and raloxifene. For example, these ligands are partial ER agonists for ERα but act as pure ER antagonists for ERβ (Barkhem, et al., 1998).

ERα and ERβ are products of distinct genes on different chromosomes. ERα is located at chromosomal locus 6q25.1 (Menasce, et al., 1993), whereas ERβ is found at position 14q22-24 (Enmark, et al., 1997).

Several ERα and ERβ splicing variants have been described. The generation of human ERα mRNA transcripts is a complex process that involves at least seven different promoters and exhibits cell line-dependent promoter usage. Most ERα variants differ only in the 5′ UTR and result in the expression of the full-length 66-kDa form of ERα (Reid, et al., 2002). Flouriot and colleagues have, however, identified a shorter 46-kDa isoform of ERα generated from an internal ATG start codon (Flouriot, et al., 2000). The shorter 46-kDa isoform of hERα lacks exon 1 and consequently the N-terminal AF-1 region (Flouriot, et al., 2000). This isoform is present in human osteoblasts and in the breast cancer cell line MCF-7, and heterodimerizes with wild-type ERα, thereby suppressing its AF-1-dependent transcriptional activity.

Several alternative splicing variants of ERβ, some with extended N-termini and others with truncations and/or insertions in the C-terminal LBD have been reported (Enmark, et al., 1997). The expression levels of many of these isoforms are higher in human breast tissues than that of wild-type ERP, and there are data supporting the protein expression of several ERβ isoforms (Fuqua, et al., 1999; Saji, et al., 2002). Expression of estrogen receptor ERβ cx protein in ERα -positive breast cancer: Specific correlation with progesterone receptor. (Saji, et al., 2002) The original human ERβ clone encoded a protein of 485 amino acids (Mosselman 1996), however, cloning of an additional N-terminal sequence has extended the N-terminus resulting in a 530 amino acid protein (Xux, et al., 2003). These proteins have been designated ERβ 1 long (530 aa) and ERP 1 short (485 aa), and the ERβ 1 long form is currently regarded as the full-length wild-type ERβ.

The 595 amino acid sequence of ERα is provided as SEQ ID NO: 8. The DBD, both the core and the CTE, is represented by amino acids 180 to 287, the sequence of which is provided as SEQ ID NO: 9. The full length 530 amino acid wild type ERβ is provided as SEQ ID NO: 10. The DBD, both core and CTE, is represented by amino acids 142 to 251, the sequence of which is provided by SEQ ID NO: 11. The nucleotide sequences of ERα and ERβ are at GenBank Accession No NM000125 and NM001437 respectively, both of which are incorporated herein by reference.

Embodiments of the present invention include polypeptide sequence that comprise SEQ ID NO: 9 or SEQ ID NO: 11, such sequences including SEQ ID NO: 8 and SEQ ID NO: 10 and fragments thereof sufficient for binding to MUC1 CD polypeptides or effective fragments thereof.

### E. Screening Methods

1. Binding Assays

The present invention provides for methods for identifying compounds that modulate, and preferably inhibit the interaction of MUC1 with ER, including ERα and ERβ. Binding is between a MUC1 polypeptide comprising the CD sequences or fragment thereof effective for binding to ER, such as sequences represented by SEQ ID NO: 6 and suitable fragments, e.g., SEQ ID NO: 7.

In one embodiment, the screening method utilizes an in vitro competitive binding assay, wherein the capacity of a test compound to inhibit the binding of a polypeptide comprising a MUC1 CD derived sequence effective for binding the supplied polypeptide comprising an ERα DBD sequence or ERβ DBD sequence effective for binding the polypeptide comprising a MUC1 CD derived sequence. In such an assay the polypeptide comprising a MUC1 CD derived sequences may comprise SEQ ID NO: 6 or SEQ ID NO: 7. The MUC1 and ER polypeptides of the invention, such as the polypeptide comprising a MUC1 CD derived sequence, may be conjugated to another protein or produced as a fusion protein, e.g., the GST-MUC1 CD and Myc-MUC1 CD fusion protein exemplified herein. Other suitable conjugates and fusion proteins may be made by one of skill in the art utilizing procedures know in the art. The polypeptides comprising an ER or MUC1 CD sequence may be labeled with a radioisotope or fluorescent label (e.g., phycobiliproteins, such as phycoerythrin and allophycocyanins, fluorescein and Texas red). Alternatively an enzyme, such as peroxidase, may be used and conjugated either directly or indirectly via a biotin and avidin or streptavidin system. Such polypeptide may be immobilized on a suitable immobile phase, such as the surface of a multiwell plate, microbead, or column packing. Suitably tagged polypeptides may are measured by methods generally known in the art. Decreased binding upon introduction of a test compound as compared to a suitable control is indicative of an agent that inhibits or otherwise decreases the binding of MUC1 CD to ER. Suitable antibodies specific for an epitope on the polypeptide comprising an MUC CD derived sequence or a polypeptide comprising an ERα DBD sequence or ERβ DBD sequence can also be utilized in suitable binding assays. Thus suitable ELISA assays as generally known in the art may be suitably utilized. The in vitro screening assays may also further comprise effective amounts of 17β-estradiol (E2).

2. In silico screening

Screening for small molecule antagonists of the association of MUC1 CD with ER can be conducted by in silico analysis. The term “in silico” refers to any method or process performed using a computer. The term “in silico analysis” refers to any type of assay or analysis of molecular interactions performed on a computer, such as in silico analysis include in silico screening, high-throughput in silico screening, in silico binding, in silico docking, in silico affinity determination, in silico molecular modeling, in silico annealing, in silico lead identification, in silico lead optimization, in silico ADMET, and the like.

In silico analysis of the binding of small molecules to a protein binding site by use of clustering conformational variants of the binding site are disclosed in US Patent Application 2004/0015299, incorporated herein by reference in its entirety. Methods for the generation of virtual combinational libraries and methods for in silico docking are disclosed in U.S. Pat. No. 6,253,168, incorporated by reference in its entirety. In silico methods include generating a set of conformational variants of the MUC1 CD ER binding region and forming a plurality of clusters of related conformational variants within the set using a clustering algorithm, and selecting a representative structure from each of the plurality of clusters.

The set of conformational variants is obtained from empirical data, such as a crystal structure, an NMR structure, or on other empirically-determined data. The method will comprise the use of a clustering algorithm, which may be based on partitioning around medoids; or use “fuzzy” or hierarchical clustering. In silico analysis may utilize in silico screening, in silico docking, in silico lead discovery, and in-silico lead optimization. For example, the in silico analysis may include screening a plurality of ligands against the molecule. The method encompasses assessing the activity (e.g., binding activity, docking activity, etc.) of a plurality of ligands on a target molecule.

Interactions of small molecule ligands with the MUC1 CD ER binding site can be studied using one or more of the existing molecular docking programs. Examples of such programs include, but are not limited to: AMBER (www.amber.ucsf.edu/amber/amber.html-), AMMP (http://www.cs.gsu.edu/.about.-cscrwh/ammp/ammp.html), CHARMM (www.yuri.harvard.edu/), Dalton Quantum Chemistry Program (www.kjemi.uio.no/software/dalton/dalton.html), Deep Viewer (www.expasy.cbr.nrc.ca/spdbv/), FTDock (www.bmm.icnet.uk/dockin-g/), TINKER (www.dasher.wustl.edu/tinker/) and the like. The representation of the MUC1 CD binding site is treated as a fixed structure, against which the commercially available docking program performs its functions directed at determining the nature of an interaction between a given ligand and a given protein.

One aspect of the invention provides for a method of producing a computer readable database comprising the three-dimensional molecular structural coordinates of the ER binding pocket of MUC1 cytoplasmic domain said method comprising a) obtaining three-dimensional structural coordinates defining said binding pocket of said protein, from a crystal of said MUC1 cytoplasmic domain; and b) introducing said structural coordinates into a computer to produce a database containing the molecular structural coordinates of said binding pocket.

Other embodiments of the invention provide for a method of producing a computer readable database containing the three-dimensional molecular structural coordinates of a compound capable of binding the ER binding site of MUC1 cytoplasmic domain, said method comprising a) introducing into a computer program a computer readable database, b) generating a three-dimensional representation of the active site or binding pocket of said MUC1 cytoplasmic domain in said computer program; c) superimposing a three-dimensional model of at least one binding test compound on said representation of the active site or binding pocket; d) assessing whether said test compound model fits spatially into the binding pocket of said MUC1 cytoplasmic domain.

Further embodiments include methods for determining whether a compound binds the MUC1 cytoplasmic domain, comprising, a) providing a computer modeling program with a set of structural coordinates or a three dimensional conformation for a molecule that comprises a binding pocket of a crystalline MUC1 cytoplasmic domain; b) providing a said computer modeling program with a set of structural coordinates of a chemical entity; c) using said computer modeling program to evaluate the potential binding or interfering interactions between the chemical entity and said binding pocket; and d) determining whether said chemical entity potentially binds to or interferes with said MUC1 cytoplasmic domain.

Embodiments also include methods of producing a computer readable database comprising a representation of a compound capable of binding a binding pocket of the MUC1 cytoplasmic domain, said method comprising a) introducing into a computer program a computer readable database; b) determining a chemical moiety that interacts with said binding pocket; c) computationally screening a plurality of compounds to determine which compound(s)comprise said moiety as a substructure of said compound(s); and d) storing a representation of said compound(s) that comprise said substructure into a computer readable database.

A used in the forgoing embodiments, the term “MUC1 cytoplasmic domain” encompasses MUC1 proteins or fragments thereof that comprise the cytoplasmic domain sequence or a fragment thereof capable of binding to an estrogen receptor DNA binding domain and also includes an isolated MUC1 cytoplasmic domain sequence or fragment thereof capable of binding to an estrogen receptor DNA binding domain.

3. Indirect assays

The evaluation of small molecules as antagonists of the association of MUC1 CD with ER can also be conducted by evaluating down stream effects resulting from the binding of MUC1 to ER. Such assays may suitably be conducted in cell free or cell systems. Suitable parameters for measurement include ER stability, ER binding to ERE and activation of estrogen-responsive genes. Such assays may also be sued as confirmatory assays for ligands identified by binding assays or in silico analysis.

### E. Antibodies

The term “antibody” is used in the broadest sense and specifically covers monoclonal antibodies (including full length monoclonal antibodies), polyclonal antibodies, multispecific antibodies (e.g., bispecific antibodies), and antibody fragments, so long as they exhibit the desired biological activity.

Methods for generating polyclonal antibodies are well known in the art. Briefly, a polyclonal antibody is prepared by immunizing an animal with an immunogenic composition and collecting antisera from that immunized animal. A wide range of animal species can be used for the production of antisera including rabbit, mouse, rat, hamster, guinea pig and goat. The serum for an immunized animal may be used as is for various applications or the desired antibody fraction may be purified by well-known methods, such as affinity chromatography using another antibody or a peptide bound to a solid matrix.

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed polypeptide. The immunizing composition is administered in a manner that effectively stimulates antibody producing cells, which may comprise, but is not limited to, administration of MUC1-CD derived peptides or transgenic cells expressing a MUC1-CD.

The methods for generating monoclonal antibodies (MAbs) generally begin along the same lines as those for preparing polyclonal antibodies. The use of rats may provide certain advantages (Goding, 1986), but mice are preferred, with the BALB/c mouse being the most routinely used and generally gives a higher percentage of stable fusions. Human antibodies may be prepared from immunized xenomice as described by U.S. Pat. No. 6,075,181 and U.S. Pat. No. 6,150,584, both incorporated herein by reference.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred. Often, a panel of animals will have been immunized and the spleen of animal with the highest antibody titer will be removed for obtaining lymphocytes from the spleen.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and have enzyme deficiencies that render them incapable of growing in certain selective media that support the growth of only the desired fused cells (hybridomas). Selected hybridomas are serially diluted and cloned into individual antibody-producing cell lines, which can then be propagated indefinitely to provide MAbs.

In accordance with the present invention, fragments of the monoclonal antibody of the invention can be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention can be synthesized using an automated synthesizer, or by expression of full-length gene or of gene fragments in E. coli or other recombinant microorganisms and cell lines.

The present invention also encompasses various antibody conjugates. Labeled conjugates are useful in various screening and diagnostic uses such as flow cytometry, immunohistochemistry and immuno-quantification methods such as ELISA techniques. Labels used in making versions of the antibodies of the present invention suitable for screening and diagnostic uses include moieties that may be detected directly, such as fluorochromes and radiolabels, as well as moieties, such as enzymes, that must be reacted or derivatized to be detected. Examples of such labels are ³²P, ¹²⁵I, ³H, ¹⁴C, fluorescein and its derivatives, rhodamine and its derivatives, dansyl, umbelliferone, luciferia, 2,3-dihydrophthalazinediones, horseradish peroxidase, alkaline phosphatase, lysozyme, and glucose-6-phosphate dehydrogenase. The antibodies may be tagged with such labels by known methods. For instance, coupling agents such as aldehydes, carbodiimides, dimaleimide, imidates, succinimides, bis-diazotized benzadine and the like may be used to tag the antibodies with the above-described fluorescent, chemiluminescent, and enzyme labels. The antibodies may also be labeled with magnetic beads for use in magnetic sorting regimens.

Conjugates with radionuclides are also useful in the preparation of therapeutic antibodies. Radionuclides useful in such conjugates include ¹³¹I, ⁹⁰Y, ¹⁰⁵Rh, ⁴⁷Sc, ⁶⁷Cu, ²¹²Bi, ²¹¹At, ¹⁸⁸Re, ¹⁰⁹Pd, ⁴⁷Sc, ²¹²Pb, and ¹⁵³Sm and the like, as described in Gansow, 1991, herein incorporated by reference. Therapeutic antibodies of the invention can also be coupled to conventional chemotherapeutic agents such as an antimetabolite, an anthracycline, a vinca alkaloid, an antibiotic, or an alkylating agent. Drugs that may be coupled to the antibodies for targeting include compounds such as doxorubicin, cyclophosphamide, cisplatin, adriamycin, estramustine, fluorouracil, ethinyl estradiol, mitoxantrone, methotrexate, finasteride, taxol, and megestrol. Methods of coupling may be direct via covalent bonds, or indirect via linking molecules, and will generally be known in the art for the particular drug selected and are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bisazido compounds such as his (R-azidobenzoyl) hexanediamine, bisdiazonium derivatives such as bis-(R-diazoniumbenzoyl)ethylenediamine, diisocyanates such as tolylene 2,6-diisocyanate, and bis-active fluorine compounds such as 1,5-difluoro-2,4-dinitrobenzene. (See, Thorpe et al., 1982, herein incorporated by reference).

“Single-chain FV” or “sFv” antibody fragments of the present invention comprise the VH and VL domains of antibody, wherein these domains are present in a single polypeptide chain. Generally, the Fv polypeptide further comprises a polypeptide linker between the VH and VL domains that enables the sFv to form the desired structure for antigen binding (see Pluckthun, 1994).

G. Antagonists of MUC1-CD Association with ER

Aspects of the present invention include antagonists of the association of MUC1-CD with ER. Such antagonists include compounds identified by the screening methods disclosed herein. Antagonists also include antibodies that bind to the relevant sites of MUC1-CD and ER-DBD that comprise the respective binding sites for the association between the two polypeptides. Thus, suitable MUC1-CD polypeptide fragments include SEQ ID NO: 7 and other such fragments disclosed herein. Other antagonists include polypeptides that comprise the respective binding sites for the MUC1-CD association with ER. Such peptides may suitable compete with a wild-type ligand. Thus, the MUC1-CD fragments such as SEQ ID NO: 7 and other fragments disclosed herein may be suitably used. Competing peptides may also comprise a suitable MUC1-CD fragment or ER-DBD fragment and an internalizing peptide sequence, i.e. a peptide that increases the transmembrane transport of the relevant MUC-1 CD fragment or ER-DBD fragment. Such internalizing peptides may suitably comprise at least a portion of the Antennapedia protein or homolog thereof (Derossi et al., 1994; Console et al., 2003; Pescarolo et al, 2001) the HIV transactivator (TAT) protein (Wender et al., 2000; Futaki et al., 2001), galanin or mastoparan chimera sequences (Pooga et al., 1998; Soomets et al., 2000), or other suitable internalizing sequence.

### H. Formulations and Treatment

Compounds that modulate the association of MUC1 and ER can be formulated in a variety of conventional pharmaceutical formulations and administered to cancer patients, in need of treatment, by any one of the drug administration routes conventionally employed including oral, intravenous, intraarterial, parental or intraperitoneal.

For oral administration the compositions of the present invention may be formulated, for example, with an inert diluent or with an assimiable edible carrier, or enclosed in hard or soft shell gelatin capsules, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, a gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit for is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compounds sucrose as a sweetening agent methyl and propylparabens as preservatives, a dye and flavoring, such as cherry or orange flavor. Of course, any material used in preparing a dosage unit form should be pharmaceutically pure and substantially non-toxic in the amounts employed. In addition, other chemotherapeutic compounds may be incorporated into sustained-release preparation and formulations.

In regard to formulations comprising oligonucleotides, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure.

Pharmaceutical formulations of the compositions of the present invention which are suitable for injectable use include sterile aqueous solutions or dispersions and sterile powders for the extemporaneous preparation of sterile injectable solutions or dispersions. In all cases the form must be sterile and must be fluid to the extent that each syringability exists. It must be stable under the conditions of manufacture and storage and must be preserved against the contaminating action of microorganisms, such as bacteria and fungi. The carrier can be a solvent or dispersion medium containing, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compositions of the present invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the composition.

Tumors that can be suitably treated with the methods of the present invention are those that estrogen sensitive. In a typical embodiment, the tumor is a breast tumor.

The treatment with the compounds of the present invention may precede, follow, or be used concomitantly with anti-estrogen treatments, such as tamoxifen.

I. Combination with Chemotherapeutic Agents

The present invention encompasses the use of the compounds of the present invention in combination with chemotherapeutic agents in addition to the anti-estrogen compounds. In this regard, the compositions of the present invention will be useful for the treatment cancer cells resistant to chemotherapeutic agents, including residual cancers remaining or reoccurring after cancer chemotherapy. The foregoing rational also pertains to the combination of compositions of the present invention and ionizing radiation.

The chemotherapeutic agents useful in the methods of the invention include the full spectrum of compositions and compounds which are known to be active in killing and/or inhibiting the growth of cancer cells. The chemotherapeutic agents, grouped by mechanism of action include DNA-interactive agents, antimetabolites, tubulin interactive agents, anti-hormonals, anti-virals, ODC inhibitors and other cytotoxics such as hydroxy-urea. Any of these agents are suitable for use in the methods of the present invention. Compositions of the present invention can also be used in combination with antibodies to HER-2, such as Trastuzumab (Herceptin (H)). In addition, the present invention also encompasses the use of compounds of the present invention with epidermal growth factor receptor-interactive agents such as tyrosine kinase inhibitors. Tyrosine kinase inhibitors suitably include imatinib (Norvartis), OSI-774 (OSI Pharmaceuticals), ZD-1839 (AstraZeneca), SU-101 (Sugen) and CP-701 (Cephalon).

When used in the treatment methods of the present invention, it is contemplated that the chemotherapeutic agent of choice can be conveniently used in any formulation which is currently commercially available, and at dosages which fall below or within the approved label usage for single agent use.

J. Combination with Ionizing Radiation

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art. The amount of ionizing radiation needed in a given cell generally depends on the nature of that cell. Means for determining an effective amount of radiation are well known in the art. Used herein, the term “an effective dose” of ionizing radiation means a dose of ionizing radiation that produces cell damage or death when given in conjunction with the SPX polypeptides of the present invention, optionally further combined with a chemotherapeutic agent.

Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Any suitable means for delivering radiation to a tissue may be employed in the present invention, in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

### EXAMPLES

### Example 1

### Experimental Procedures

Cell culture. MCF-7 and MDA-MB-231 breast cancer cells and COS-1 cells were grown in Dulbecco's modified Eagle's medium (DMEM) with 10% heat-inactivated fetal bovine serum (HI-FBS), 100 mg/ml streptomycin, 100 units/ml penicillin and 2 mM L-glutamine. Human ZR-75-1 breast cancer cells and those stably infected with a control retroviral vector (ZR-75-1/vector) or one expressing a MUC1siRNA (ZR-75-1/MUC1siRNA) (Ren, et al., 2004) were cultured in RPMI 1640 medium supplemented with 10% HI-FBS, 100 mg/ml streptomycin, 100 units/ml penicillin and 2 mM L-glutamine. Cells were grown in phenol red-free medium supplemented with 10% charcoal-dextran-stripped FBS for 3 d before treatment with E2 (Sigma, St. Louis, Mo.), TAM (Sigma), MG132 (Peptides International Inc.) or ICI182,780 (ICI; Tocris, Washington, D.C.).

Coimmunoprecipitation and immunoblotting. Lysates prepared from subconfluent cells as described (Wei, et al., 2003) were subjected to immunoprecipitation with anti-ERα (D-12; Santa Cruz Biotechnology), anti-Myc (Ab-1; Oncogene Research Products) or anti-PCNA (F-2; Santa Cruz Biotechnology). Immunoblot analysis was performed with anti-MUC1-C (Ab-5; NeoMarkers), anti-ERα, anti-β-actin (Sigma), anti-Ub (Santa Cruz Biotechnology), anti-SRC-1, anti-GRIPI (Upstate Biotechnology Inc.), anti-pS2 (Santa Cruz Biotechnology), anti-cathepsin D (E-7; Santa Cruz Biotechnology) or anti-PCNA. Immunocomplexes were detected with enhanced chemiluminescence (ECL; PerkinElmer Life Sciences).

Plasmid construction and transfection. PCR products prepared from pIRESpuro2-MUC1 (Li, et al., 2001b) as a template were cloned into pCMV-Myc (Invitrogen) to generate Myc-MUC1-CD. The PCR products were also digested with BamHI/NotI and cloned into corresponding sites of pGEX-4T-3 to generate GST-MUC1-CD. Vectors expressing ERa were generated by PCR using pcDNA3.1-ERa (Shao, et al., 2002) as a template and cloning of the products into the BamHI and XhoI sites of pcDNA3.1 (Invitrogen). Transient transfections for coimmunoprecipitation studies were performed in 60-mm dishes using Fugene-6 (Roche Applied Science). MDA-MB-231 cells were transfected with pIRESpuro2 or pIRESpuro2-MUC1 and selected in the presence of puromycin (Calbiochem-Novabiochem). Single cell clones were isolated by limiting dilution and expanded for analysis.

GST pull-down assays. ERa was labeled with ³⁵S in in vitro transcription/translation (TNT) reactions (Promega) and incubated with purified GST or GST-MUC1-CD fusion proteins for 2 h at 4° C. After washing, the adsorbed proteins were resolved by SDS-PAGE and detected by phosphoimaging (Molecular Dynamics).

Reporter assays. MCF-7 and ZR-75-1 cells grown in phenol red-free medium supplemented with 10% charcoal-dextran-stripped FBS were transfected with ERE-tk-Luc (Shao et al., 2002) and ERα using the calcium phosphate method (Invitrogen). At 18 h after transfection, cells were treated with E2 or TAM for 24 h and assayed for luciferase activity using the Luciferase Assay System (Promega). Luciferase activity was normalized for transfection efficiency using a control LacZ vector (pCMV-LacZ) (Wei, et al., 2001).

Chromatin immunoprecipitation (ChIP). ChIP assays were performed as described (Shang, et al., 2000). For Re-ChIP assays, complexes from the primary ChIP were eluted with 10 mM DTT for 30 min at 370C, diluted 1:30 in 1% 20 mM Tris-HCl, pH 8.1, Triton X-100, 2 mM EDTA, 150 mM NaCl, and reimmunoprecipitated with anti- ERa, anti-SRC-l (1135; Upstate Biotechnology Inc.) or anti-GRIP 1 (CT; Upstate Biotechnology Inc.) antibodies. For PCR, 2 ml from a 50 ml DNA extraction were used with 25-35 cycles of amplification.

Pulse-chase experiments. Cells were washed twice with methionine-free medium and then incubated in methionine-free medium containing 50 mCi/ml [³⁵S]-methionine (New England Nuclear) for 60 min at 37° C. The cells were then washed and cultured in medium containing 10% charcoal-dextran-stripped FBS and 10 nM E2. At different times during the chase, cells were harvested and lysates were immunoprecipitated with anti-ERα. The precipitates were analyzed by SDS-PAGE and autoradiography.

Colony formation assays. Aliquots of 500 cells/well were grown in 6 well plates containing 2 ml/well phenol red-free medium supplemented with 10% charcoal-dextran-stripped FBS for 24 h at 37° C. The cells were treated with 100 nM E2, 1 mM TAM or 100 nM ICI for 10 d. The resulting colonies were stained with crystal violet and counted manually.

### Example 2

### MUC1 associates with ERα

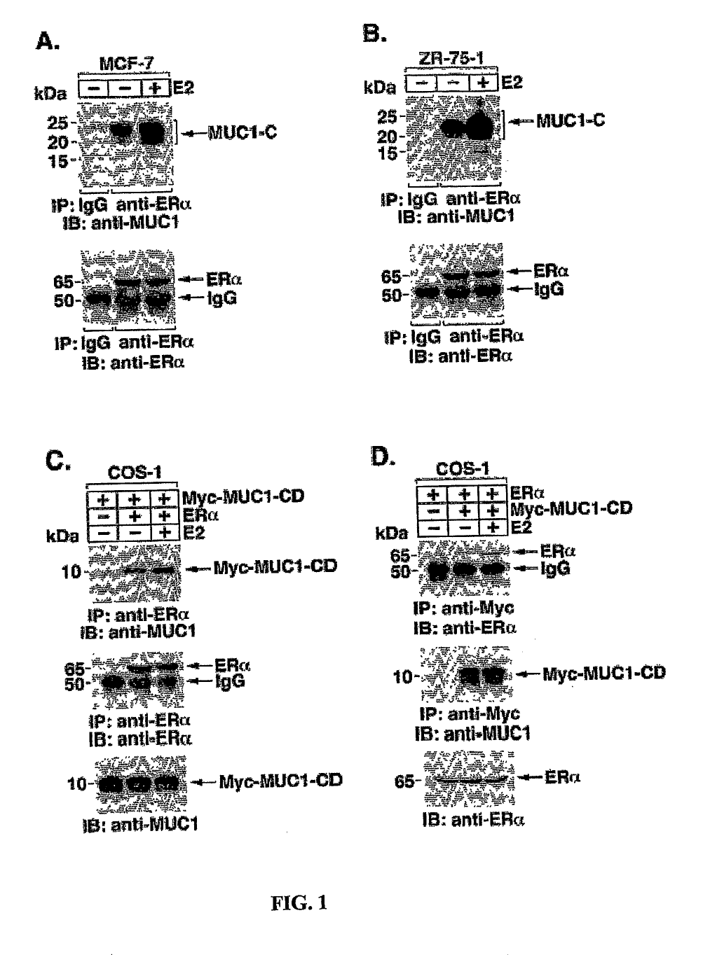

Human MCF-7 breast cancer cells were studied to determine if MUC1 interacts with ERα. In MCF-7 and other cell types, the MUC1 C-terminal subunit (MUC1-C) is expressed as a ˜20-25 kDa protein and to a lesser extent as ˜17-12 kDa fragments. Immunoblot analysis of anti-ERα immunoprecipitates with an antibody that reacts with the MUC1 cytoplasmic domain (MUC1-CD) demonstrated that ERα coprecipitates with MUC1-C (FIG. 1A). As a control, there was no detectable MUC1-C in precipitates prepared with IgG (FIG. 1A). The results also demonstrate that the association between ERα and MUC1-C is increased by 17β-estradiol (E2) stimulation (FIG. 1A). Similar studies performed with ZR-75-1 breast cancer cells confirmed that ERα associates with MUC1-C and that the association is stimulated by E2 (FIG. 1B). Densitometric scanning of the MUC1 signals obtained from whole cell lysates as compared to that after immunoprecipitation of the lysates with anti-ERα indicate that ˜3 and 5% of the total MUC1-C associates with ERα in control and E2-stimulated MCF-7 cells, respectively. In ZR-75-1 cells, 4 and 6% of total MUC1-C associated with ERα in the absence and presence of E2, respectively. As a control, cell lysates were immunoprecipitated with an antibody against the proliferating cell nuclear antigen (PCNA). There was no detectable MUC1-C in the anti-PCNA precipitates from MCF-7 or ZR-75-1 cells. To define the region of MUC1-C responsible for the interaction, Myc-tagged MUC1 cytoplasmic domain (Myc-MUC1-CD) was coexpressed with ERα in COS-1 cells. Immunoblot analysis of anti-ERα precipitates with anti-MUC1-CD demonstrated that MUC1-CD is sufficient for the association with ERα (FIG. 1C). Moreover, stimulation of the COS-1 cells with E2 increased binding of ERα and Myc-MUC1-CD (FIG. 1C). This association was confirmed in the reciprocal experiment in which anti-Myc immunoprecipitates were immunoblotted with anti-ERα (FIG. 1D). These findings indicate that MUC1 associates with ERα constitutively and that this interaction is increased in the response to E2.

### Example 3

### MUC1-CD Binds Directly to ERα

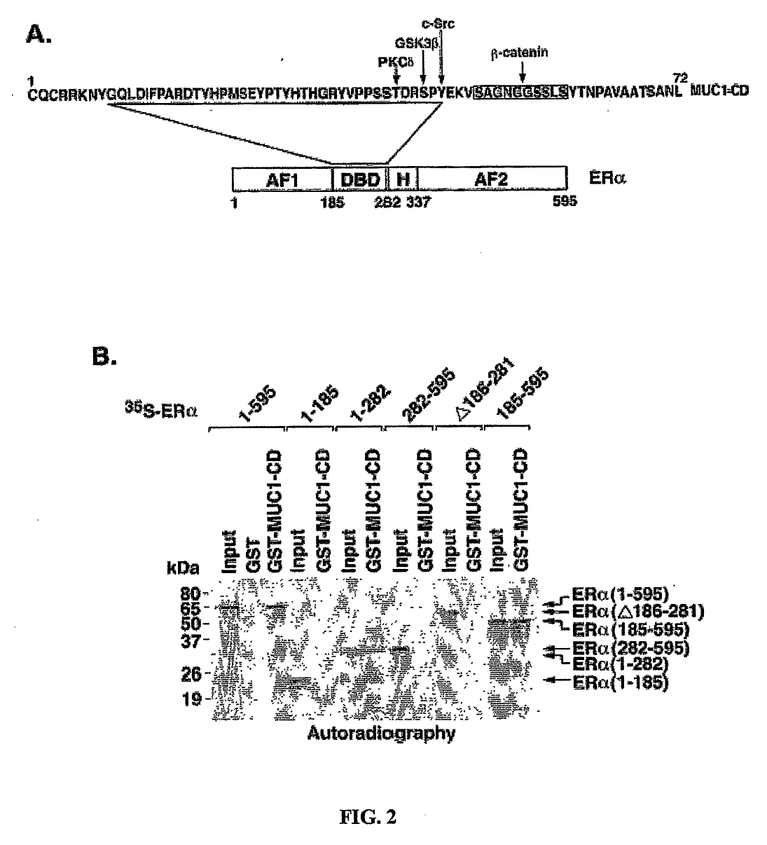

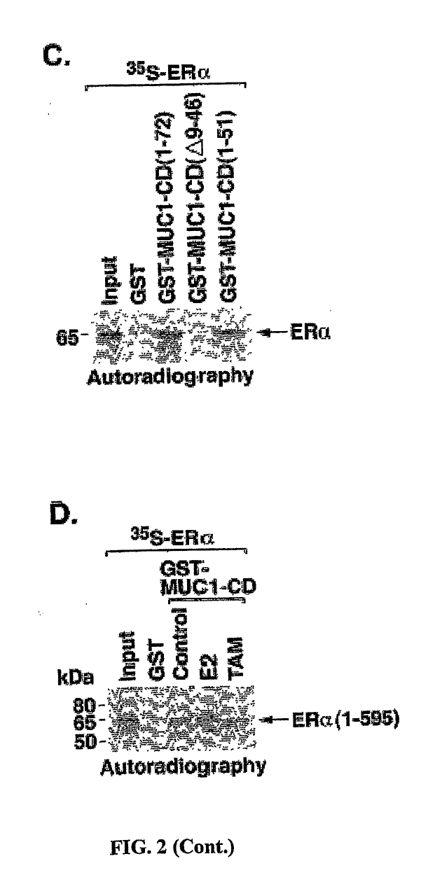

To define the regions of MUC1-CD (72 amino acids) and ERa (595 amino acids) responsible for the interaction (FIG. 2A), GST or a GST-MUC1-CD fusion protein was incubated with ³⁵S-labeled ERα in vitro. Analysis of adsorbates to glutathione beads demonstrated binding of full-length ERα(1-595) to GST-MUC1-CD and not GST (FIG. 2B). By contrast, there was no detectable binding of MUC1-CD to ERα(1-185) that contains the AF1 domain (FIG. 2B). Moreover, the demonstration that MUC1-CD binds to ERα(1-282) indicated involvement of the DNA binding domain (DBD) (FIG. 2B). Consistent with these results, binding of MUC1-CD was found with ERα(185-595), but not ERα (282-595) or ERα(Δ186-281) devoid of the DBD (FIG. 2B). To localize the region within MUC1-CD that interacts with ERα, ³⁵S-labeled full length ERα was incubated with deletion mutants of MUC1-CD. The results demonstrate that ERα binds to both full-length MUC 1-CD and MUC1-CD(1-51), indicating that the N-terminal region of MUC1-CD is sufficient for the interaction (FIG. 2C). Consistent with those results, deletion of MUC1-CD amino acids 9 to 46 abrogated the association with ERα (FIG. 2C). Binding in vitro was also compared in the absence and presence of E2. The results show an E2-dependent increase in the binding of MUC1-CD and full-length ERα (FIG. 2D). By contrast, TAM had no apparent effect on the formation of MUC1-CD-ERα complexes (FIG. 2D). These findings indicate that MUC1-CD(9-46) binds directly to the ERα DBD and that this interaction is stimulated by E2.

### Example 4

### MUC1 Occupies Estrogen-Responsive Gene Promoters

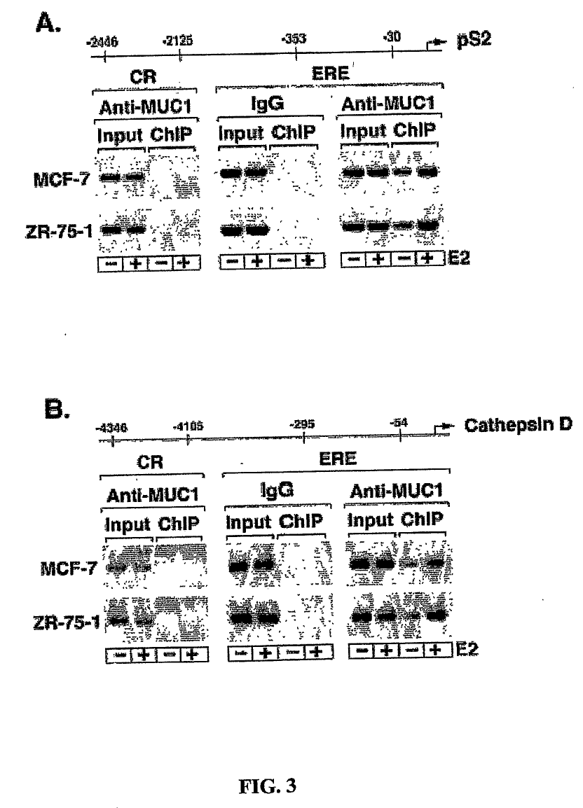

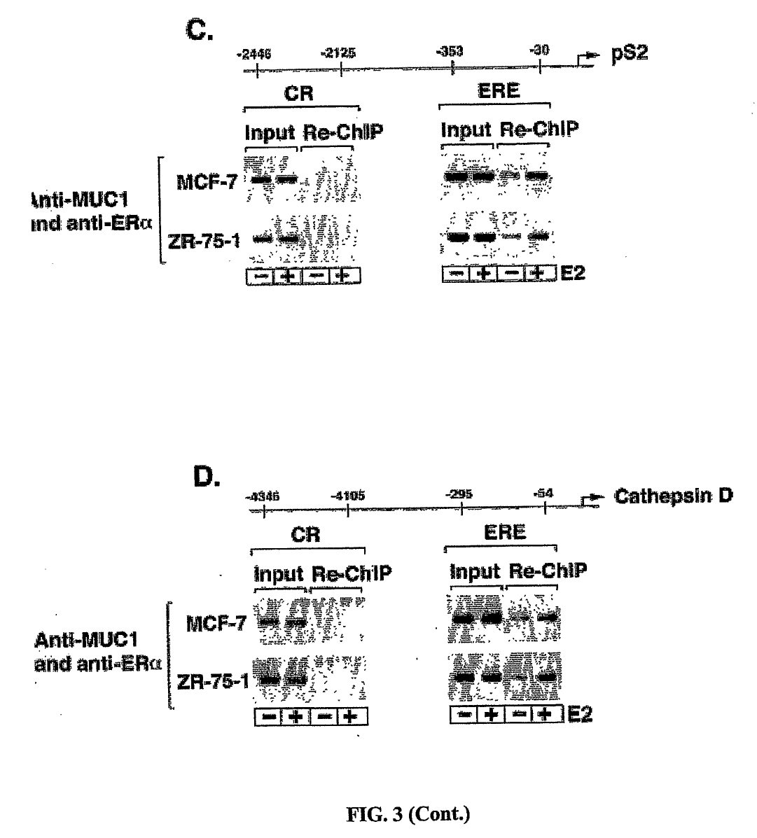

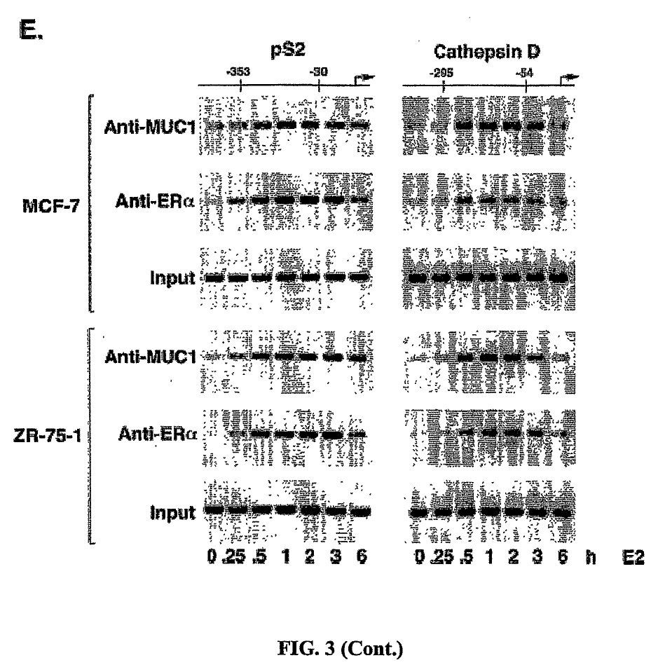

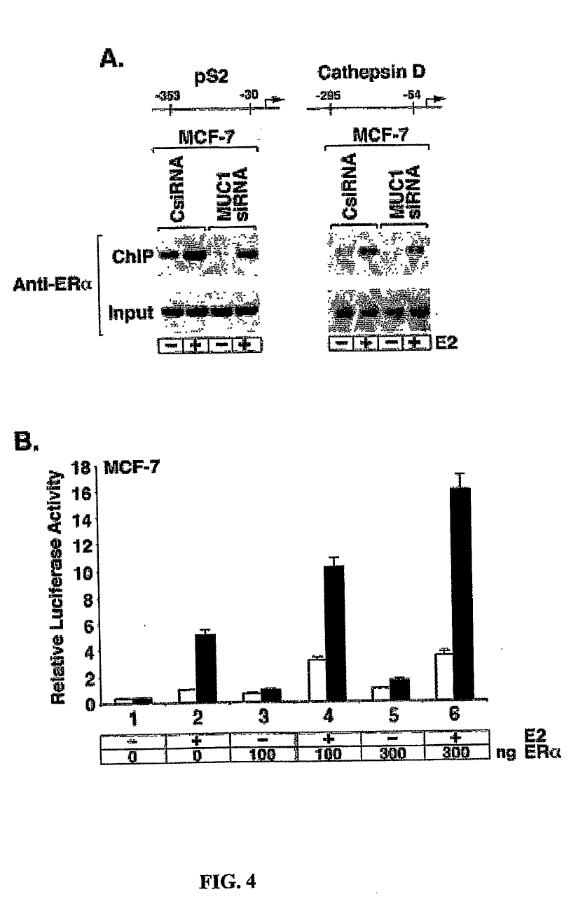

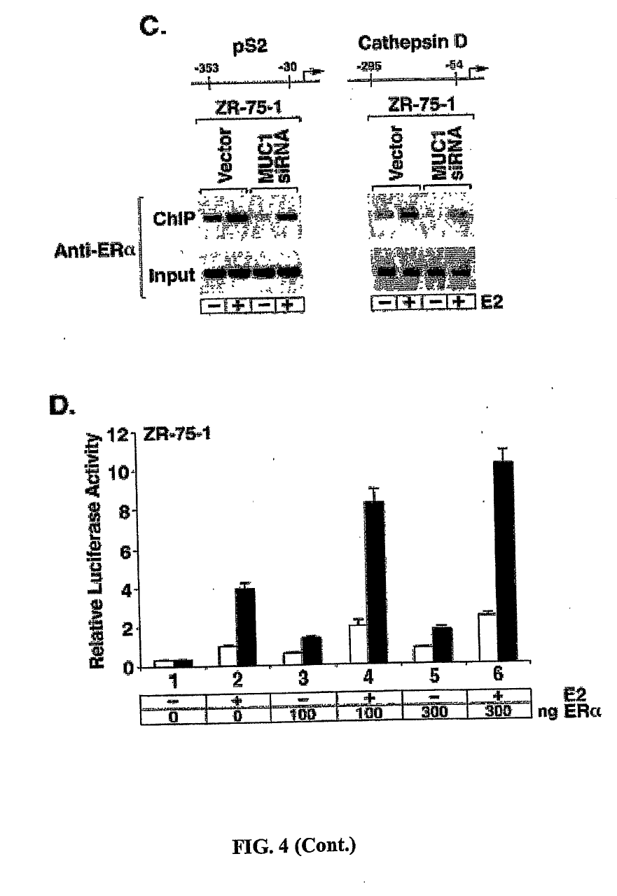

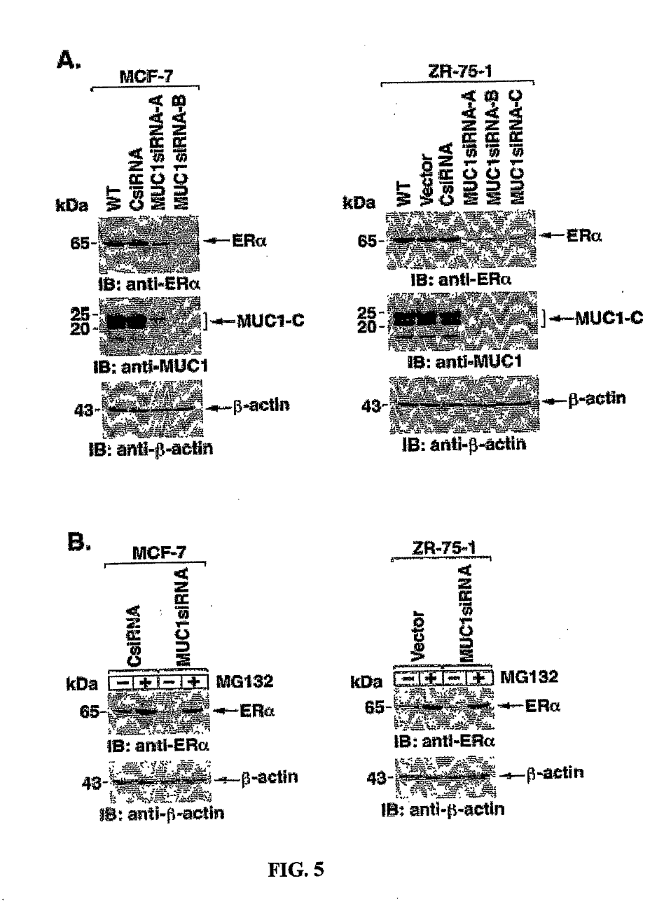

To determine if MUC1 is present in the ERα transcription complex, we performed chromatin immunoprecipitation (ChIP) assays with anti-MUC1-C. Immunoprecipitation of the estrogen responsive region (ERE) in the promoter of the pS2 gene (-353 to -30) (Giamarchi , et al., 1999) was analyzed by semiquantitative PCR. In both MCF-7 and ZR-75-1 cells, occupancy of the pS2 promoter by MUC1 was detectable in the absence of E2 and was increased by E2 stimulation (FIG. 3A). As controls, there were no detectable pS2 promoter sequences in immunoprecipitates performed with IgG (FIG. 3A). There was also no detectable MUC1 associated with a control region (CR; −2446 to −2125) of the pS2 promoter upstream to the ERE (FIG. 3A). The chromatin immunoprecipitates were further analyzed for the estrogen-responsive region (−295 to −54) of the cathepsin D gene promoter (Augereau, et al., 1994). As found for the pS2 promoter, MUC1 occupancy of the cathepsin D promoter in MCF-7 cells was detectable constitutively and was increased by E2 stimulation (FIG. 3B). By contrast, MUC1 occupancy was not detectable in a control region (CR; −4346 to −4105) of the cathepsin D promoter (FIG. 3B). Similar results were obtained in ZR-75-1 cells (FIG. 3B). To assess whether MUC1 occupies the pS2 promoter with ERα, the anti-MUC1 complexes were released, re-immunoprecipitated with anti-ERα and then analyzed by PCR (Re-ChIP). As shown for both MCF-7 and ZR-75-1 cells, anti- ERα precipitated the pS2 promoter after release from anti-MUC1, indicating that MUC1 is present in the region occupied by the ERα transcription complex (FIG. 3C). The results also demonstrate that the cathepsin D promoter is immunoprecipitated with anti-ERα after release from anti-MUC1 (FIG. 3D). In concert with the demonstration that E2 stimulates binding of ERα and MUC1, the Re-ChIP assays further showed that E2 exposure increases complexes of ERα and MUC1 on the pS2 and cathepsin D promoters (FIGS. 3C and D). The kinetics of MUC1 occupancy of the EREs was also assessed by performing ChIPs at different intervals of E2 stimulation. Like ERα, increases in MUC 1 occupancy of the pS2 and cathepsin D EREs were detectable at 15 to 30 min of E2 exposure (FIG. 3E). Moreover, maximal occupancy for both MUC1 and ERα was observed when the cells were stimulated with E2 for 1 to 3 h (FIG. 3E). Of note and as shown previously (Metivier, et al., 2003; Metivier , et al., 2002; Reid , et al., 2003; Shang , et al., 2000), ERα occupies the pS2 and cathepsin D EREs at low but detectable levels in the absence of E2 stimulation (FIG. 3E). To determine if MUC1 occupancy of EREs is dependent on ERα, human MDA-MB-231 breast cancer cells, which are negative for MUC1 and express low levels of ERα, were stably transfected to express an empty vector or MUC1. The MDA-MB-231/MUC1 transfectants expressed MUC1 at levels comparable to that in ZR-75-1 cells. The MDA-MB-231/vector and MDA-MB-231/MUC1 cells were also transiently transfected to express ERα. Notably, compared to MCF-7 cells, MUC1 occupancy of the pS2 and cathepsin D EREs was substantially decreased in the MDA-MB-231/MUC1 cells. Moreover, MUC1 occupancy of the pS2 and cathepsin D EREs was markedly increased by transfection of ERα. These findings indicate that i) MUC1 is a component of the ERα transcription complex, ii) E2 stimulation is associated with increases in occupancy of both ERα and MUC1 on estrogen-responsive promoters, and iii) MUC1 occupancy of EREs is dependent on ERα.

### Example 5

### MUC1 Coactivates ERa-Mediated Transcription

To assess the effects of MUC1 on ERα function, MCF-7 cells were stably infected with a retrovirus expressing MUC1siRNA. Immunoblot analysis of two separately isolated clones demonstrated partial (˜80-90%) and complete down-regulation of MUC1 in MCF-7/MUC1siRNA-A and MCF-7/MUC1siRNA-B cells, respectively, as compared to that in cells expressing a control siRNA (CsiRNA). ChIP assays performed on the MCF-7/CsiRNA and MCF-7/MUC1siRNA-A cells showed that ERα occupancy of the pS2 promoter is decreased by knocking-down MUC1 expression. As expected, E2 stimulation was associated with increased occupancy of the pS2 promoter by ERa; however, this response was attenuated in the MCF-7/MUC1siRNA cells. Similar effects of MUC1 were observed when analyzing the cathepsin D promoter. To assess the effects of MUC1 on ERα-mediated transcription, the MCF-7/CsiRNA and MCF-7/MUC1siRNA-A cells were transfected with an ERE-tk-Luc reporter and then stimulated with E2. MUC1 expression was associated with little if any activation of the ERE promoter in the absence of E2 stimulation. By contrast, MUC1-dependent activation of ERE-tk-Luc was increased ˜5-fold when the cells were stimulated with E2. Further increases in MUC1-dependent stimulation of ERa-mediated transcription were found when the MCF-7 cells were transfected with different amounts of the ERα vector. To determine if MUC1 exhibits similar effects in ZR-75-1 cells, we used ZR-75-1/vector and ZR-75-1/MUC1siRNA cells (Ren, et al., 2004). Knocking-down MUC1 expression in ZR-75-1 cells decreased ERα occupancy of the pS2 promoter in both the absence and presence of E2 stimulation. Similar results were obtained when analyzing the cathepsin D ERE. Knocking-down MUC1 in ZR-75-1 cells also decreased E2-mediated activation of the ERE-tk-Luc reporter. These findings indicate that MUC1increases ERα occupancy of estrogen-responsive promoters and coactivates ERα-mediated transcription.

### Example 6

### MUC1 Stabilizes ERα

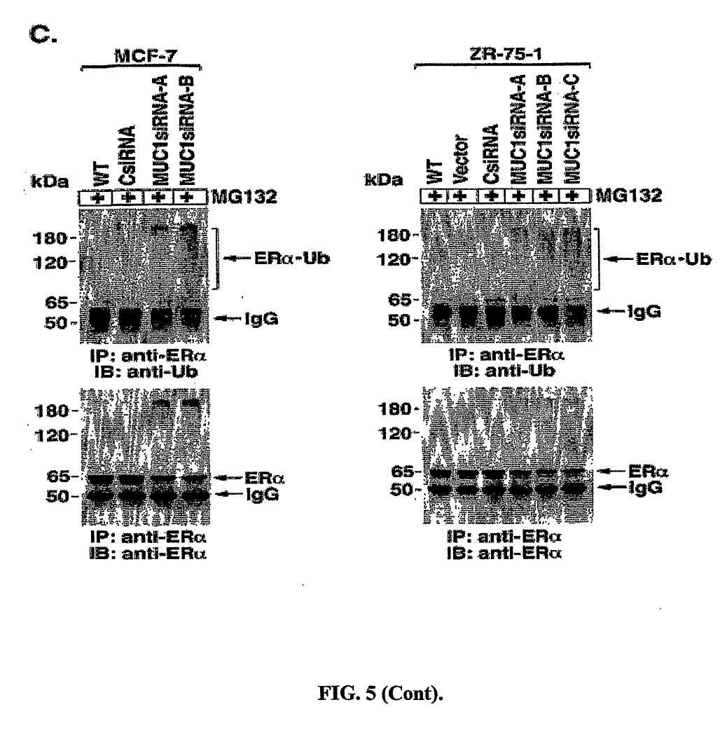

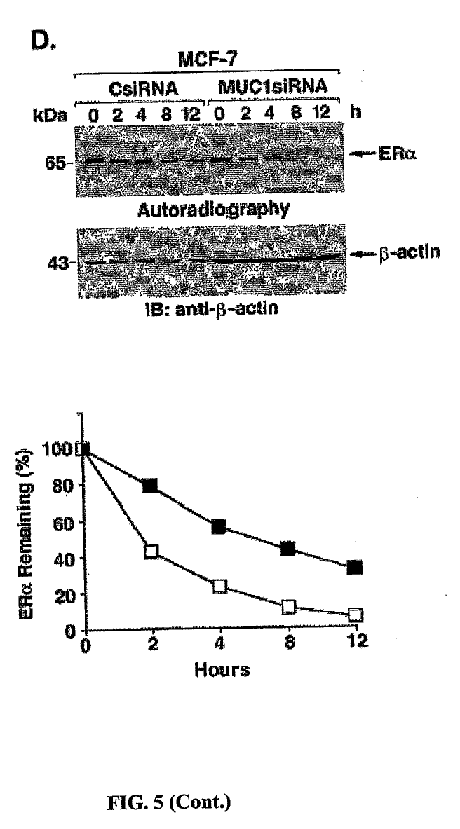

The finding that downregulation of MUC1 attenuates ERα occupancy of EREs prompted us to ask if MUC1 affects ERα expression. As such, ERα levels were compared in the MCF-7, MCF-7/CsiRNA and MCF-7/MUC1siRNA cells. The results demonstrate that knocking-down MUC1 is associated with decreases in ERα expression (FIG. 5A, left). Similar decreases in ERα levels were observed in ZR-75-1 cells expressing MUC1siRNA (FIG. SA, right). RT-PCR analysis demonstrated that ERα mRNA levels are similar in the presence and absence of MUC1 (data not shown), indicating that MUC1 regulates ERα by a post-translational mechanism. In this regard, stability of the ERa protein is controlled by ubiquitination and proteosomal degradation (Lonard , et al., 2000; Nawaz , et al., 1999; Reid , et al., 2003). Consequently, ERα levels were assessed in the response of MCF-7 and ZR-75-1 cells to the proteosomal inhibitor MG132. Inhibition of the proteosome was associated with increases in ERα expression and this effect was more pronounced in cells silenced for MUC1 (FIG. 5B). Immunoblot analysis of anti-ERα precipitates with anti-ubiquitin (Ub) further showed that downregulation of MUC1 in MCF-7 cells is associated with increased ubiquitination of ERα (FIG. 5C, left). Similar results were obtained in ZR-75-1 cells (FIG. 5C, right). To further assess the effects of MUC1 on ERα stability, cells were pulsed with [³⁵S]-methionine and ERα was immunoprecipitated at various intervals during the chase period. Analysis of ERα by autoradiography showed that the half-life of ERα is decreased in the absence of MUC1 in both MCF-7 (FIG. 5D) and ZR-75-1 (Supplemental FIG. S4A) cells. The estrogen antagonist ICI1 82,780 (ICI) targets ERα to the proteosome (Dauvois , et al., 1992; Reid, et al., 2003). Consistent with a role for MUC1 in stabilizing ERα, ICI-induced down-regulation of ERα was attenuated in the presence of MUC1 (Supplemental FIG. S4B). These findings indicate that MUC1 stabilizes ERα by blocking its ubiquitination and proteosomal degradation.

### Example 7

### MUC1 Stimulates Occupancy of Transcriptional Coactivators on Estrogen-Responsive Promoters and Attenuates the Effects of TAM

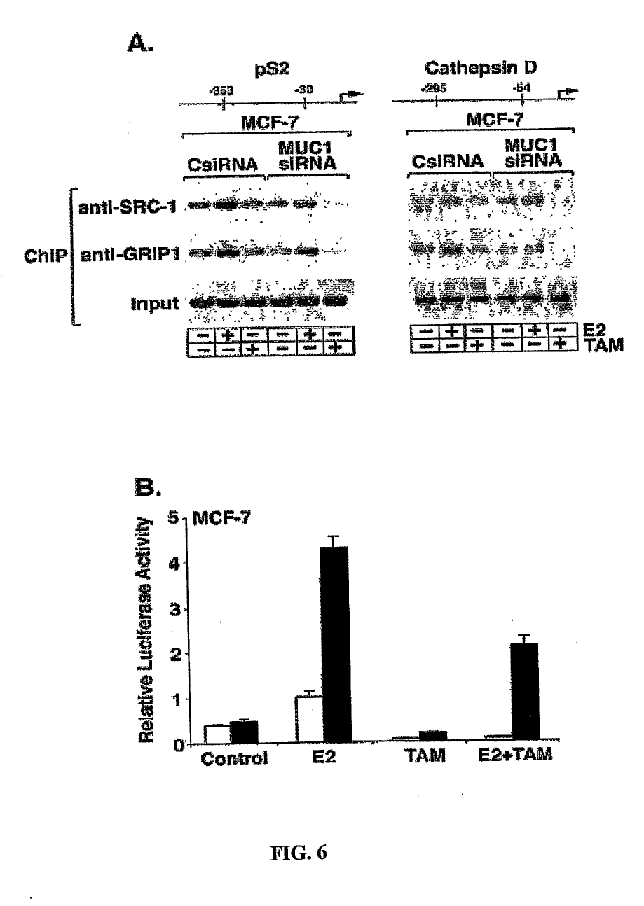

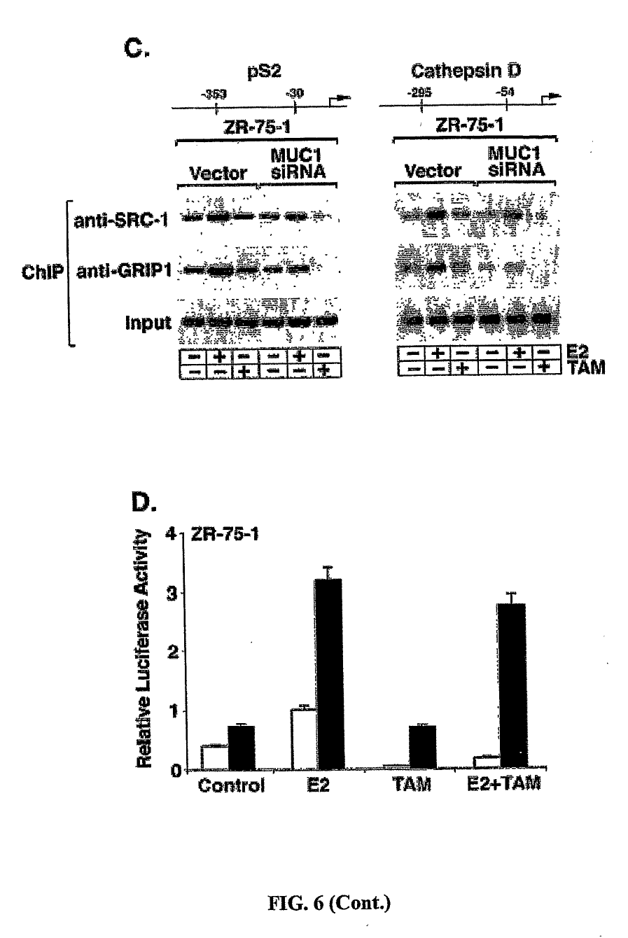

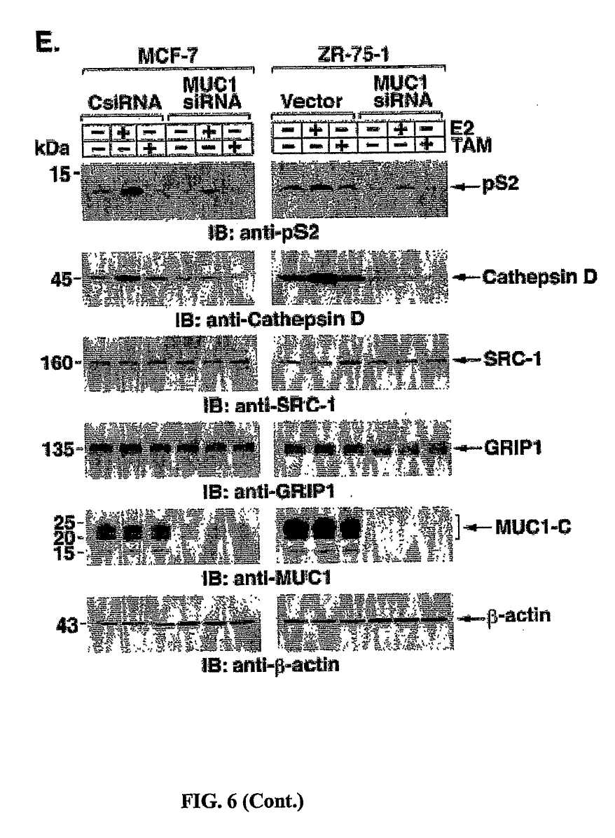

To determine if MUC1-dependent stabilization of ERα affects recruitment of transcriptional coactivators, we asked if MUC1 occupies EREs with the p160 family members, SRC-1 and GRIP-1. In Re-ChIP assays performed on MCF-7 cells, release of anti-MUC1 immunoprecipitates and re-precipitation with anti-SRC-1 demonstrated that SRC-1 is present in MUC1 complexes on both the pS2 and cathepsin D promoters. Similar results were obtained when the Re-ChIP assays were performed on soluble chromatin from ZR-75-1 cells (Supplemental FIG. S5A). Also, in both cells, promoter complexes of MUC1 and SRC-1 were increased by E2 stimulation. The results of Re-ChIP assays further showed that MUC1 associates with GRIP1 on the pS2 and cathepsin D promoters (Supplemental FIG. S5B). To further assess the effects of MUC1 on coactivator occupancy of E2-reponsive promoters, ChIP assays were performed on the MCF-7/CsiRNA and MCF-7/MUC1siRNA-A cells. Occupancy of the pS2 and cathepsin D promoter by SRC-1 was more pronounced in the MUC1-positive cells in both the absence and presence of E2 stimulation (FIG. 6A). Similar results were obtained for GRIP1 occupancy (FIG. 6A). Moreover, TAM was substantially more effective in decreasing SRC1/GRIP1 occupancy of the pS2 and cathepsin D promoters in MCF-7 cells with downregulation of MUC1 expression (FIG. 6A). Consequently, we asked if MUC1 attenuates the effects of TAM on ERα-mediated transcription. Down-regulation of E2-induced transcription by TAM was attenuated in MCF-7/CsiRNA, as compared to that in MCF-7/MUC1siRNA, cells (FIG. 6B). TAM-induced decreases of SRC-1/GRIP1 occupancy on the pS2 and cathepsin D promoters were also attenuated by MUC1 expression in ZR-75-1 cells (FIG. 6C). Moreover, down-regulation of E2-induced transcription by TAM was attenuated in ZR-75-1/vector, as compared to that in ZR-75-1/MUC1siRNA, cells (FIG. 6D). In concert with these results, E2 stimulation of pS2 and cathepsin D expression was attenuated by downregulation of MUC1 in both MCF-7 and ZR-75-1 cells (FIG. 6E). These findings indicate that MUC1 potentiates the response to E2 and antagonizes the inhibitory effects of TAM on estrogen-responsive promoters.

### Example 8

### MUC1 Attenuates Anti-Estrogen-Induced Loss of Survival

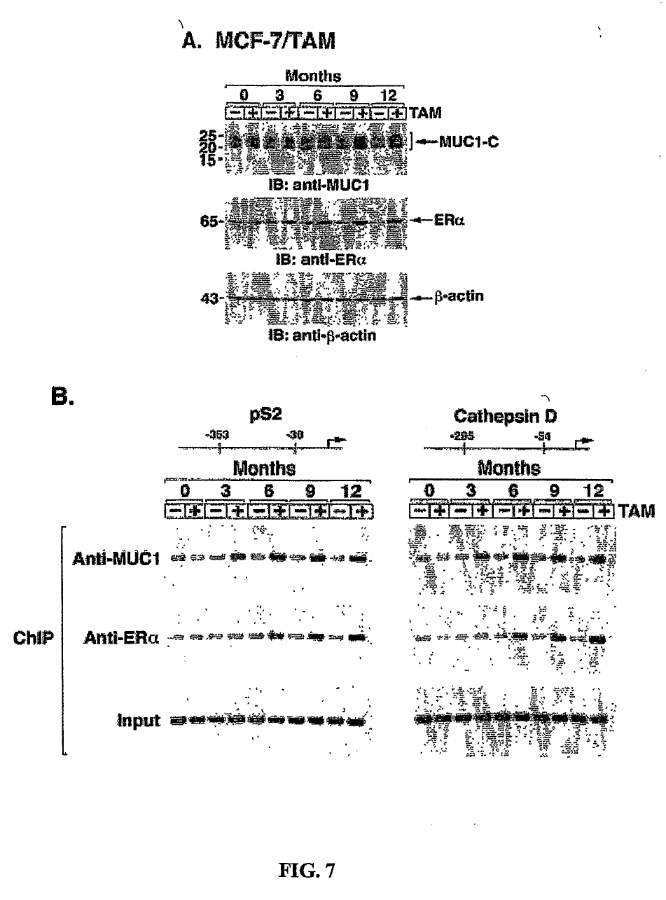

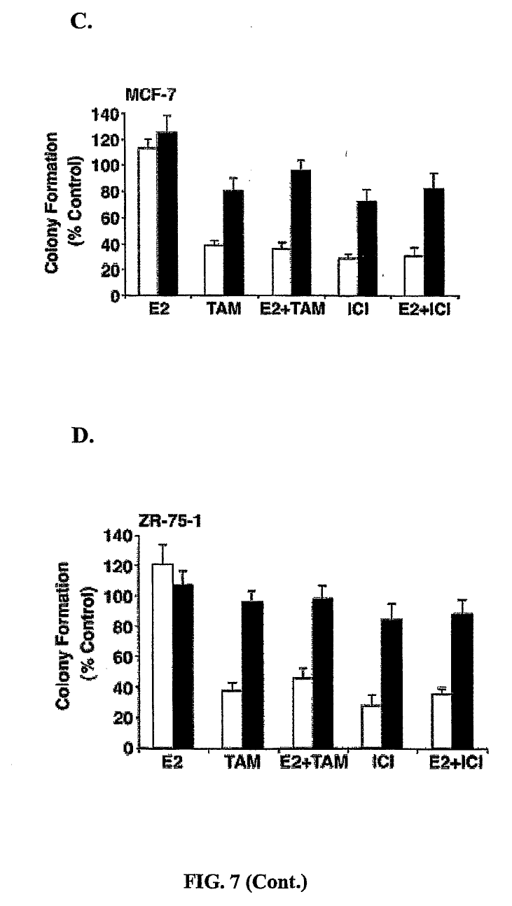

To determine if MUC1 is associated with TAM resistance, we exposed MCF-7 cells to increasing concentrations of TAM over 12 months. Little if any change in MUC1 was observed at 3-6 months; however, significant increases in MUC1 expression were detected at 9 and 12 months of TAM exposure (FIG. 7A). Consistent with a role for MUC1 in stabilizing ERa, increases in MUC1 levels were associated with upregulation of ERα (FIG. 7A). Analysis of the EREs of the pS2 and cathepsin D promoters further showed that TAM resistance is associated with increased occupancy by both MUC1 and ERα (FIG. 7B). To directly assess whether MUC1 affects the sensitivity of breast cancer cells to TAM-induced death, the exposed MCF-7/CsiRNA and MCF-7/MUC1siRNA-A cells to TAM and monitored survival by colony formation. TAM reduced the survival of MCF-7/MUC1siRNA cells by over 60% (FIG. 7C). By contrast, survival of TAM-treated MCF-7/CsiRNA cells was significantly greater in the absence or presence of E2 (FIG. 7C). Knocking-down MUC1 expression also decreased survival when MCF-7 cells were exposed to ICI (FIG. 7C). Similar results obtained with the ZR-75-1/vector and ZR-75-1/MUC1siRNA cells (FIG. 7D) provided further support for involvement of MUC1 in protecting MCF-7 and ZR-75-1 breast cancer cells against anti-estrogen-induced loss of survival.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 6,075,181
U.S. Pat. No. 6,150,584
U.S. Pat. No. 6,253,168
U.S. Publn. 2004/0015299
PCT Appln. WO9603502
Ali & Coombes, Nat. Rev. Cancer, 2:101-112, 2002.
Augereau et al., Mol. Endocrinol., 8:693-703, 1994.
Barkhem et al., Mol. Pharmacol., 54:105-112. 1998.
Beato & Klug, Hum. Reprod., 6:225-236, 2000.
Benz et al., Breast Cancer Res. Treat., 24:85-95, 1993.
Brzozowski et al., Nature, 389:753-758, 1997.
Busti, Trends Biochem. Sci., 26:152-153, 2001.
Buzdar & Hortobágyi, Clin. Cancer Res., 4:527-534, 1998.
Buzdar et al., Arimidex Study Group Cancer, 83:1142-1152, 1998.
Cavailles et al. EMBO J 14, 3741-3751, 1995.
Cameron et al. Eur J Cancer 36:845-851, 2000.
Cameron et al. Eur J Cancer 37, 1545-1553, 2001.
Chakravarti et al. Nature 383, 99-103, 1996.
Chen et al. Science 284, 2174-2177, 1999.
Chen et al. Cell 90, 569-580, 1997.
Colletti et al. Cancer Res 49, 1882-1884, 1989.
Console et al., J. Biol. Chem., 278 :35109-35114, 2003.
Couse & Korach, Endocr. Rev. 20, 358-417 (1999).
Dauvois et al. Proc Natl Acad Sci USA 89, 4037-4041, 1992.
Derossi et al., J Biol. Chem., 269:10444-10450, 1994.
Enmark et al. J. Clin. Endocrinol. Metab. 82, 4258-4265 (1997).
Flouriot et al. EMBO J. 19, 4688-4700 (2000).
Fuqua et al. Cancer Res. 59, 5425-5428 (1999).
Futaki et al., J. Biol. Chem., 276 :5836-5840, 2001.
Gendler et al. J Biol Chem 263, 12820-12823, 1988.
Giamarchi et al. Oncogene 18, 533-541, 1999.
Goding, In: Monoclonal Antibodies: Principles and Practice, 2d ed., Orlando, Fla., Academic Press, pp. 60-61, 1986.
Halachmi et al. Science 264, 1455-1458, 1994.
Honget et al. Mol Cell Biol 17, 2735-2744, 1997.
Howell et al. Brit. J Cancer, 74, 300-308, 2000.
Huang et al. Cancer Biol Ther 2, 702-706, 2003.
Ing et al. J Biol Chem 267, 17617-17623, 1992.
Jacq et al. Cell 79, 107-117, 1994.
Jordan & Dowse, J Endocrinol 68, 297-303, 1976.
Khorasanizadeh & Rastinejad, Trends Biochem. Sci. 26, 384-390, 2001.
Kiang et al. Cancer 45, 1322-1325, 1980.
Klinge, Nucleic Acids Res. 29, 2905-2919 (2001).
Kouzmenko et al. J Biol Chem 279, 40255-40258, 2004.
Krege et al. Proc Natl Acad Sci USA 95, 15677-15682, 1998.
Kufe et al. Hybridoma 3, 223-232, 1984.
Kuiper et al. Endocrinology 139 , 4252-4263 (1998).
Kurokawa et al. Cancer Res 60, 5887-5894, 2000.
Le Douarin et al. EMBO J 14, 2020-2033, 1995.
Li et al. Mol Cell Biol 18, 7216-7224, 1998.
Li et al. Cancer Biol Ther 2, 187-193, 2003a.
Li & Kufe, Biochem Biophys Res Commun 281, 440-443, 2001.
Li et al. J Biol Chem 276, 6061-6064, 2001 a.
Li et al. Oncogene 22, 6107-6110, 2003b.
Li et al. J Biol Chem 276, 35239-35242, 2001b.
Li et al. Mol Cancer Res 1, 765-775, 2003c.
Ligtenberg et al. J Biol Chem 267, 6171-6177, 1992.
Lonard et al. Mol Cell 5, 939-948, 2000.
Lubahn et al. Proc Natl Acad Sci USA 90, 11162-11166, 1993.
Mangelsdorf et al. Cell 83, 835-839, 1995.
Melvin et al. J Biol Chem 279, 14763-14771, 2004.
Melvin et al. J Biol Chem 277, 25115-25124, 2002.
Merlo et al., Cancer Res 49, 6966-6971, 1989.
Metivier et al. Cell 115, 751-763, 2003.
Metivier et al. Mol Cell 10, 1019-1032, 2002.
Mosselman et al. FEBS Lett. 392, 49-53 (1996).
Muss et al. J Clin Oncol 6, 1098-1106, 1988.
Nawaz et al. Proc Natl Acad Sci USA 96, 1858-1862, 1999.
Nicholson et al. Endocr Relat Cancer 8, 175-182, 2001.
Nilsson et al. Physiol. Rev. 81, 1535-1565 (2001).
Oesterreich et al. Mol Endocrinol 14, 369-381, 2000.
Onate et al. Science 270, 1354-1357, 1995.
Oosterkamp et al., Int. J Cancer, 72:87-94, 1997.
Osborne et al. Cancer Res 43, 3583-3585, 1983.
Paech et al. Science 277, 1508-1510 (1997).
Pereyetal. Cancer Res 52, 2563-3568, 1992.
Pescarolo et al., FASEB J., 15 :31-33, 2001.
Pettersson & Gustafsson, Annu. Rev. Physiol. 63 , 165-192 (2001).
Pluckthun in The Pharmacology of Monoclonal Antibodies, vol. 113:269-315, 1994.
Pooga et al., Nature Biotech., 16 :857-861, 1998.
Rachez et al. Nature 398, 824-828, 1999.
Rachez & Feedman, Curr. Opin. Cell Biol. 13, 274-280 (2001).
Ravdin et al. J Clin Oncol 10, 1284-1291, 1992.
Reid et al. Mol Cell 11, 695-707, 2003.
Reid et al. Cell. Mol. Life Sci. 59, 821-831 (2002).
Ren et al. Cancer Cell 5, 163-175, 2004.
Romine et al. Mol Endocrinol 12, 664-674, 1998.
Saji et al. Cancer Res. 62 , 4849-4853 (2002).
Schroeder et al. J Biol Chem 276, 13057-13064, 2001.
Schroeder et al. Oncogene 23, 5739-5747, 2004.
Schwabe et al. Cell 74, 57-578, 1993.
Senkus & Edwards, Steroids 64, 576-586, 1999.
Shang et al. Cell 103, 843-852, 2000.
Shao et al. Mol Cell Biol 22, 3358-3372, 2002.
Shao et al. Proc Natl Acad Sci USA 101, 11599-11604, 2004.
Shiau et al. Cell 95, 927-937, 1998.
Siddiqui et al. Proc Natl Acad Sci USA 85, 2320-2323, 1988.
Smith et al., Mol Endocrinol 11, 657-666, 1997.
Spencer et al., Nature 389, 194-198, 1997.
Soomets et al, Biochim. Biophys. Acta, 1467:165-176, 2000.
Sun et al. Endocrinology 140, 800-804 (1999).
Swallow et al., Nature 328, 82-84.
Thorpe et al., Immunological Rev. 62:119-158, 1982.
Thorpe et al., Monoclonal Antibodies in Clinical Medicine, pp. 168-190 Academic Press, NY 1982(a).
Vermeer et al. Nature 422, 322-326, 2003.
vom Baur et al. EMBO J 15, 110-124, 1996.
Webb et al. Mol Endocrinol 12, 1605-1618, 1998.
Wei et al. J Biol Chem 276, 16107-16112, 2001.
Wei et al. Cancer Cell 7, 167-178, 2005.
Wei et al. J Biol Chem 278, 29288-29297, 2003.
Wen et al. J Biol Chem 278, 38029-38039, 2003.
Wender et al., Proc. Nat'l. Acad. Sci., U.S.A., 97:13003-13008, 2000
Yamamoto et al. J Biol Chem 272, 12492-12494, 1997.
Yin & Kufe. J Biol Chem 278, 35458-35464, 2003.
Yuan et al. Proc Natl Acad Sci USA 95, 7939-7944, 1998.
Xux et al. Endocrinology 144, 3541-3546 (2003).
Yao & Jordan, The Oncologist 3, 104-110, 1998.
Zhang et al. Cancer Res 57, 1244-1249, 1997.
Zilliacus et al. Mol. Eidocrinol. 9, 389-400, 1995.
Zrihan-Licht et al., Eur. J. Biochem., 224:787-795, 1994.

1. A method of screening a compound for effectiveness of modulating the association of a MUC1 cytoplasmic domain polypeptide with an estrogen receptor comprising:
providing a first polypeptide comprising SEQ ID NO 7 or a fragment thereof, capable of binding to an ER DNA binding domain;
providing a second polypeptide comprising SEQ ID NO: 9, SEQ ID NO: 11, or a fragment thereof, capable of binding to MUC1 CD;
providing a candidate compound;
quantifying the association between said first and said second polypeptide; and
comparing said quantification of the association between said first and said second polypeptide with an appropriate control.

2. The method of claim 1, wherein said second polypeptide comprises SEQ ID NO: 9 or a fragment thereof, wherein the fragment is capable of binding to MUC1 CD.

3. The method of claim 1, wherein said second polypeptide comprises SEQ ID NO: 11 or a fragment thereof, wherein the fragment capable of binding to MUC1 CD.

4. The method of claim 1. wherein said first polypeptide is immobilized by linkage to a stationary phase.

5. The method of claim 1. wherein said second polypeptide is immobilized by linkage to a stationary phase.

6. The method of claim 1, wherein said first polypeptide further comprises a fluorescent label, a radiolabel, or a chromophore.

7. The method of claim 1, wherein said second polypeptide further comprises a fluorescent label, a radiolabel, or a chromophore.

8. The method of claim 1, wherein said first polypeptide is a fusion protein.

9. The method of claim 1, wherein said second polypeptide is a fusion protein.

10. The method of claim 1 further comprising proving 17β-estradiol in an amount sufficient to enhance the association of said first and said second polypeptide.

11. A method of screening for a compound that specifically binds to a MUC1 cytoplasmic domain polypeptide comprising SEQ ID NO 7 or a fragment thereof, capable of binding to an estrogen receptor DNA binding domain, the method comprising: a) combining said polypeptide with at least one test compound under suitable conditions, and b) detecting binding of the test compound to polypeptide, thereby identifying a compound that specifically binds to said polypeptide.

12. A method for screening for compounds effective for preventing or inhibiting anti-estrogen resistance comprising identifying a compound that decreases the association of the MCU1 cytoplasmic domain to an estrogen receptor.


SEQUENCE LISTING (12 sequences)

SEQ ID NO: 1 - protein, 1344 aa, Homo sapiens
MTPGTQSPFFLLLLLTVLTATTAPKPATVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 2 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 3 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEASSRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYETVSAGNGGSSLSYTNPAVAATSANL

SEQ ID NO: 4 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYETVSAGNGGSSLSYTNPAVAATSANL

SEQ ID NO: 5 - protein, 230 aa, Homo sapiens
MTPGTQSPFFLLLLLTVLTVVTGSGHASSTPGGEKETSATQRSSVPSSTEKNAIPAPTTTKSCRETFLKWPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 6 - protein, 72 aa, Homo sapiens
CQCRRKNYGQLDIFPARDTYHPMSEYPTYHTHGRYVPPSSTDRSPYEKVSAGNGGSSLSYTNPAVAATSANL

SEQ ID NO: 7 - protein, 38 aa, Homo sapiens
GQLDIFPARDTYNPMSEYPTYHTHGRYVPPSSTDRSPY

SEQ ID NO: 8 - protein, 595 aa, Homo sapiens [sequence omitted]

SEQ ID NO: 9 - protein, 108 aa, Homo sapiens
KETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGMDR

SEQ ID NO: 10 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 11 - protein, 110 aa, Homo sapiens
SKRDAHFCAVCSDYASGYHYGVWSCREGCKAFFKRSIQGHNDYCPATNQCTIDKNRRKSCQACRLRKCYEVGMVKCGSRRERCGYRLVRRQRSADEQLHCAGKAKRSGGH

SEQ ID NO: 12 - protein, 17 aa, Homo sapiens
IPAPTTTKSCRETFLKW